(19) United States
(12) Patent Application Publication
(10) Pub. No.: US 2025/0325319 A1
(43) Pub. Date: Oct. 23, 2025

(54) TRANSCATHETER OR MINIMALLY INVASIVE SURGERY SYSTEM AND METHODS OF USING SAME

(71) Applicant: MEACOR, INC., Irvine, CA (US)

(72) Inventors: Toufic Azar, Irvine, CA (US); Omar Labban, Irvine, CA (US); Anthony Eid, Irvine, CA (US); Renzo Cecere, Irvine, CA (US)

(21) Appl. No.: 19/171,567

(22) Filed: Apr. 7, 2025

Related U.S. Application Data
(63) Continuation of application No. PCT/US23/07629, filed on Oct. 6, 2023.
(60) Provisional application No. 63/378,866, filed on Oct. 8, 2022.

(51) Int. Cl. A61B 18/02; A61B 18/00; A61F 2/24

(52) U.S. Cl. CPC A61B 18/02; A61F 2/2466; A61B 2018/00095; A61B 2018/00184; A61B 2018/00369; A61B 2018/0212; A61B 2018/0262; A61F 2220/0008

(57) ABSTRACT

The present disclosure relates to a transcatheter surgery system and methods for performing mitral valve annuloplasty and other surgical procedures. In an aspect, the transcatheter system includes a driving catheter configured to drive one or more anchors into tissue, a cinching catheter configured to cinch a wire extending through the one or more anchors, and a cutting catheter configured to cut a portion of the wire. The present disclosure also relates to an implant kit including a helical anchor defining an anchor passageway extending therethrough, a beaded wire defining longitudinally spaced apart beads, a stopper, the stopper being larger than the central passageway so as to be prevented from passing therethrough, and a cinching element larger than the central passageway so as to be prevented from passing therethrough, the cinching element defining a one-way gap allowing passage of the wire therethrough in only one direction.

### RELATED APPLICATION DATA

This application is a continuation of International application No. PCT/US2023/07629, filed Oct. 6, 2023, which claims benefit of U.S. provisional application Ser. No. 63/378,866, filed Oct. 8, 2022, the entire disclosures of which application are expressly incorporated herein by reference.

### BACKGROUND

The present disclosure relates to the general fields of surgery and medical devices and, more particularly, to a transcatheter or minimally invasive surgery system and methods of using same.

Many medical conditions can be treated by joining two pieces of tissue together or by reducing the dimensions of an opening. Examples of such conditions include cardiac valve regurgitation and wounds wherein two pieces of tissue need to be maintained in contact with each other until the natural healing process is completed. To perform these procedures, access to the surgery site is required, which can be problematic in many cases due to limitation in space. For example, open heart surgery is extremely risky for the patient. While some surgeries are performed through catheter, laparoscopy, endoscopy and similar modalities, the devices come with a steep learning curve as they are complex and challenging for the operator.

Thus, there is a need on the market for improved devices and methods for performing surgical treatments that avoids the aforementioned disadvantages, all-the-while being simple, robust and reproducible. An object of the present disclosure is therefore to provides such improved devices and methods.

### SUMMARY

In a broad aspect, there is provided a cutting catheter for cutting a wire, the cutting catheter comprising: an elongated wire receiving member provided with a wire receiver at a wire receiving member distal end thereof for receiving the wire; an elongated cutting member provided with an annular blade at a cutting member distal end thereof, the annular blade being positionable adjacent the wire receiver; the cutting member being longitudinally movable relative to the wire receiving member and axially rotatable relative thereto so that the blade can both translate and rotate relative to a wire received in the wire receiver.

In another broad aspect, there is provided a method of cutting a wire using a tubular cutting member provided with a distal annular blade, the method comprising: positioning the annular blade to abut against the wire at a contact location with the wire entering the cutting member; and axially rotating the annular blade to cut through the wire.

In yet another broad aspect, there is provided a cinching system usable with a beaded wire defining beads, comprising a cinching element including an attachment; and a stopper configured for allowing movements of the beaded wire therethrough in a distal to proximal direction and prevent movements of the beaded wire therethrough in a proximal to distal direction; and a cinching catheter including a distally provided cinching element holder for receiving the attachment; and a cinching element lock for reversibly locking the attachment and the cinching element holder to each other.

In yet another broad aspect, there is provided a cinching element defining proximal and distal ends, the cinching element being usable with a cinching catheter and a beaded wire, the cinching element comprising: a proximally located attachment selectively attachable to the cinching catheter; and a distally located stopper configured for allowing movements of the beaded wire therethrough in a distal to proximal direction and prevent movements of the beaded wire therethrough in a proximal to distal direction.

In yet another broad aspect, there is provided a cryoadhesion procedure catheter assembly, comprising: a tubular body supporting a hollow thermally transmissive element distally relative thereto, the thermally transmissive element including a flexible portion bendable to conform to a predetermined shape; a pulling assembly secured distally to the thermally transmissive element and including a pull wire extending along the body, the pulling assembly being configured for bending the flexible portion; and a cooling fluid supply for supplying a cooling fluid to the thermally transmissive element.

In yet another broad aspect, there is provided a transcatheter system, comprising: a driving catheter configured to drive one or more anchors into tissue; a cinching catheter configured to cinch a wire extending through the one or more anchors; and a cutting catheter configured to cut a portion of the wire.

In yet another broad aspect, there is provided an implant kit, comprising: a helical anchor defining an anchor passageway extending therethrough; a beaded wire defining longitudinally spaced apart beads; a stopper, the stopper being larger than the central passageway so as to be prevented from passing therethrough; and a cinching element larger than the central passageway so as to be prevented from passing therethrough, the cinching element defining a one-way gap allowing passage of the wire therethrough in only one direction.

In yet another broad aspect, there is provided an implant kit, comprising: at least two helical anchors defining each an anchor passageway extending therethrough; a beaded wire defining longitudinally spaced apart beads; a cinching element defining a one-way gap allowing passage of the wire therethrough in only one direction.

In yet another broad aspect, there is provided a wound closing device, comprising: a cryoadhesion device including a hollow thermally conductive element and a cooling fluid supply for supplying a cooling fluid thereto; and an anchor driver for driving a helical anchor over the thermally conductive element.

Aspects of the disclosure herein provide a cutting catheter for cutting a wire, the cutting catheter including a wire receiving member including a wire receiver at a distal end of the wire receiving member, the wire receiver configured to receive the wire, a cutting member including an annular blade at a distal end of the cutting member, the annular blade being positionable adjacent the wire receiver, and the cutting member being longitudinally movable relative to the wire receiving member and axially rotatable relative thereto, where the annular blade is configured to both translate and rotate relative to the wire. In some embodiments, the cutting member is biased in a distally oriented direction relative to the wire receiver.

In some embodiments, longitudinal movements and axial rotations of the cutting member relative to the wire receiving member are independent from each other. In some embodiments, the wire receiving member and cutting member are flexible. In some embodiments, the wire receiving member and cutting member are bendable to be able follow a patient's vasculature. In some embodiments, the wire receiver includes a wire aperture extending transversally therethrough configured to receive the wire therethrough. In some embodiments, the wire receiving member defines a wire receiving member passageway extending longitudinally therethrough and opening in the wire aperture; and where the wire receiving member and cutting member are concentric. In some embodiments, the system further includes a tensioner configured to apply a tension to the wire. In some embodiments, the cutting member further includes a tubular member surrounding at least part of the wire receiving member.

In some embodiments, the system further includes an actuating assembly, the actuating assembly including a body, the wire receiving member being mounted to the body, and a cutting member actuator operatively coupled to the cutting member for selectively and independently rotating and longitudinally moving the cutting member relative to the body. In some embodiments, the cutting member is movable between proximal and distal positions relative to the body, the cutting member being lockable in translation relative to the body in the proximal position. In some embodiments, the system further includes a biasing element provided between the cutting member and the body for biasing the cutting member towards the distal position.

In some embodiments, the cutting member actuator includes a cutting member mount, where the cutting member is configured to extend through the cutting member mount and be jointly axially rotatable therewith, and a knob mounted to the body to be axially rotatable relative thereto, the knob defining a knob aperture extending axially therethrough receiving the cutting member mount so that the cutting member mount is axially movable along the knob and jointly rotatable with the cutting member mount.

Aspects of the disclosure herein provide a method of cutting a wire using a tubular cutting member provided with a distal annular blade, the method including positioning an annular blade to abut against a wire at a contact location with the wire entering a tubular cutting member, and axially rotating the annular blade to cut through the wire. In some embodiments, the method further includes pushing the annular blade towards the wire. In some embodiments, the method further includes pushing the annular blade towards the wire includes biasing the annular blade with a biasing element. In some embodiments, the annular blade is rotated over at least one full turn before the wire is cut. In some embodiments, the wire is pinched between the cutting blade and a member fixed relative to the annular blade. In some embodiments, the method further includes applying a tension on the wire. In some embodiments, the method is performed inside a body of a patient.

Aspects of the disclosure herein provide a cinching system usable with a beaded wire defining beads, including a cinching element including an attachment and a stopper configured for allowing movements of the beaded wire therethrough in a distal to proximal direction and prevent movements of the beaded wire therethrough in a proximal to distal direction, and a cinching catheter including a distally provided cinching element holder for receiving the attachment, and a cinching element lock for reversibly locking the attachment and the cinching element holder to each other.

In some embodiments, the stopper includes a tubular body and leaves provided inside the tubular body and converging towards each other inside the stopper in a direction leading proximally, the leaves being movable between narrow and wide configurations, a central gap between the leaves being smaller in the narrow configuration than in the wide configuration so that the beads are movable through the gap in the wide configuration but prevented from moving through the gap in the narrow configuration. In some embodiments, the leaves extend integrally as a single piece of material from the tubular body. In some embodiments, the leaves are made of a Ni—Ti shape memory alloy. In some embodiments, in the wide configuration, the leaves are biased towards the narrow configuration.

In some embodiments, the cinching catheter is adapted to selectively open the gap to allow both distally and proximally oriented movements of the beaded wire therethrough. In some embodiments, the tubular body includes circumferentially spaced apart sectors, the sectors being deformable between undeformed and an open configuration where, in the open configuration the gap larger than in the undeformed configuration, the cinching catheter includes a sector actuator for selectively moving the sectors to the open configuration.

In some embodiments, the cinching element holder and the attachment are complementarily shaped so that when the cinching element holder and the attachment are mounted to each other relative longitudinal and circumferential movements between the cinching element holder and the attachment are prevented, and unless the attachment is locked to the cinching element holder, the attachment is free to move laterally in at least one direction relative to the cinching element holder. In some embodiments, the attachment and cinching element holder are both hollow, and the cinching element lock includes an elongated member selectively movable between extended and retracted positions, where in the extended position, the cinching element lock extends through both the cinching element holder and the attachment, and in the retracted position, the cinching element lock is retracted proximally relative to the attachment so that the latter is free to move laterally relative to the cinching element holder.

In some embodiments, the attachment and cinching element holder are both internally threaded and the cinching element lock is externally threaded, so that in the extended position, the cinching element lock threadedly engages the attachment and cinching element holder. In some embodiments, the stopper is hollow and includes deformable leaves extending internally and configured to allow passage of the beads through the stopper in a proximally leading direction while preventing passage of the beads through the stopper in a distally leading direction.

In some embodiments, the stopper includes a tubular stopper body defining circumferential sectors separated from each other by slits, each leaf extending from a single sector, the cinching element lock is hollow and movable distally to engage the sectors to flare the sectors outwardly and separate the leaves from each other to create a gap allowing the beads to move distally therethrough.

In some embodiments, the cinching element lock is tubular and selectively movable between extended and retracted positions, where in the extended position, the cinching element lock surrounds at least part of both the cinching element holder and the attachment to prevent lateral movements therebetween, and in the retracted position, the cinching element lock is retracted proximally relative to the attachment so that the latter is free to move laterally relative to the cinching element holder. In some embodiments, the leaves together form a conical shape tapering proximally.

Aspects of the disclosure herein provide a cinching element defining proximal and distal ends, the cinching element being usable with a cinching catheter and a beaded wire, the cinching element including a proximally located attachment selectively attachable to the cinching catheter; and a distally located stopper configured for allowing movement of the beaded wire therethrough in a distal to proximal direction and prevent movement of the beaded wire therethrough in a proximal to distal direction.

In some embodiments, the stopper includes a tubular body and leaves provided inside the tubular body and converging towards each other inside the stopper in a direction leading proximally, the leaves being movable between a narrow configuration and a wide configuration, a central gap between the leaves being smaller in the narrow configuration than in the wide configuration so that the beads are movable through the gap in the wide configuration but prevented from moving through the gap in the narrow configuration.

In some embodiments, the leaves extend integrally as a single piece of material from the tubular body. In some embodiments, where in the wide configuration, the leaves are biased towards the narrow configuration. In some embodiments, the tubular body defines circumferentially spaced apart sectors, the leaves being each supported by one of the sectors, the sectors being deformable to an open configuration where the gap is enlarged to allow movement of the beads in the proximal to distal direction.

Aspects of the disclosure herein provide a cryoadhesion procedure catheter assembly, including a tubular body supporting a hollow thermally transmissive element distally relative thereto, the thermally transmissive element including a flexible portion bendable to conform to a predetermined shape, a pulling assembly secured distally to the thermally transmissive element and including a pull wire extending along the body, the pulling assembly being configured for bending the flexible portion, and a cooling fluid supply for supplying a cooling fluid to the thermally transmissive element.

In some embodiments, the system further includes a guiding member extending inside the flexible portion, the guiding member being stiffer in bending in a first plane than in a second plane orthogonal to the first plane, both the first and second planes extending along the bellow, where the guiding member limits bending in the first plane when the pull wire is pulled while allowing bending in the second plane. In some embodiments, the guiding member includes a plate extending laterally across the flexible member and longitudinally along at least part of the flexible member. In some embodiments, the plate is perforated.

In some embodiments, the cooling fluid supply defines a fluid outlet, the cooling fluid supply being movable relative to the thermally transmissive portion so that the cooling fluid outlet is positionable at different longitudinal positions there along. In some embodiments, including an anchor driving catheter for driving a helical anchor over the thermally transmissive portion.

In some embodiments, the tubular body defines an anchor engagement proximally to the thermally transmissive portion, the anchor engagement being configured for engaging the anchor and constraining movements of the anchor there along to a helical movement. In some embodiments, the anchor engagement prevents the anchor driving catheter from advancing distally further than the anchor engagement. In some embodiments, the anchor engagement includes at least two protrusions extending radially outwardly from the tubular body circumferentially and longitudinally offset relative to each other. In some embodiments, the anchor engagement includes a helical flange protruding radially outwardly from the tubular body.

In some embodiments, the system further includes further including an anchor catheter actuator for selectively advancing and rotating the anchor driving catheter relative to the tubular body. In some embodiments, advancement and rotation of the anchor driving catheter are independent from each other. In some embodiments, advancement and rotation of the anchor driving catheter are lockable independently from each other. In some embodiments, the anchor catheter actuator includes a casing and a knob mounted to the casing, the knob defining an axial knob passageway receiving the driving catheter therethrough, the driving catheter being secured to the knob to be jointly axially movable and rotatable relative thereto. In some embodiments, the knob is mounted to a knob mount to be jointly longitudinally movable therewith and axially rotatable relative thereto, the knob mount being mounted to the casing to be longitudinally movable there along and fixed in axial rotation relative thereto.

In some embodiments, the system further includes a rotation lock for selectively locking a relative rotation between the knob mount and the knob and a translation lock for selectively locking a relative translation between the casing and the knob mount. In some embodiments, the casing defines a longitudinally elongated mount cavity receiving the knob mount thereinto and a pair of slits extending longitudinally there along between outside of the casing and the mount cavity, the translation and rotation locks including respectively a translation lock threaded fastener and a rotation lock threaded fastener each extending radially through a respective one of the slits so as to be longitudinally movable there along and engaging a respective threaded aperture formed in the knob mount, where when the translation and rotation lock threaded fasteners are fully threaded in their respective threaded aperture, translation and rotation of the knob relative to the casing are respectively locked.

Aspects of the disclosure herein provide a transcatheter system, including a driving catheter configured to drive one or more anchors into tissue, a cinching catheter configured to cinch a wire extending through the one or more anchors, and a cutting catheter configured to cut a portion of the wire. In some embodiments, the system further includes a cooling catheter and a cooling system for providing a cooling fluid to the cooling catheter, where the cooling catheter is configured to cryoadhere to a portion of the tissue. In some embodiments, the cooling catheter includes a thermally transmissive portion having a flexible portion, the flexible portion being bendable to conform to a shape of a valve annulus. In some embodiments, when bent, the flexible portion extends over between about 135 and about 225 degrees.

In some embodiments, the system further includes a tensioning device for applying tension to the wire. In some embodiments, the wire defines longitudinally spaced apart beads. In some embodiments, the system further includes at least one helical anchor mountable to the driving catheter for being driven in the tissue. In some embodiments, the system further includes a stopper and a cinching element, the stopper and cinching element being both larger than a central passageway of the helical anchor, the wire being securable to the stopper and the cinching element being configured to allow passage of the wire therethrough only in one direction. In some embodiments, the at least one anchor is configured to be implanted at a mitral valve annulus along a portion of the mitral valve selected from the group consisting of: from the P1 area to the A1 area, from the P2 area to the A3 area, from the P1 area to the P3 area, at the P1 area, at the P2 area, at the P3 area, from the P3 to the A3 area. In some embodiments, the at least one anchor includes from 2 to 30 coils. In some embodiments, the at least one anchor is configured to span between about 45 degrees and about 225 degrees along the mitral valve annulus. In some embodiments, the at least one anchor is configured to span between about 225 degrees and about 315 degrees along the mitral valve annulus.

In some embodiments, the cinching catheter includes a double shell construction including two layers axially rotatable relative to each other and configured for receiving the wire therebetween.

In some embodiments, the cooling and driving catheter are the cryoadhesion procedure catheter assembly as defined in any one of the embodiments described above, the cutting catheter is one in any one of the embodiments described above, and the cinching catheter is as defined in any one of the embodiments described above.

Aspects of the disclosure herein provide an implant kit, including a helical anchor defining an anchor passageway extending therethrough, a beaded wire defining longitudinally spaced apart beads, a stopper, the stopper being larger than the central passageway so as to be prevented from passing therethrough, and a cinching element larger than the central passageway so as to be prevented from passing therethrough, the cinching element defining a one-way gap allowing passage of the wire therethrough in only one direction. In some embodiments, the stopper is securable to the beaded wire. In some embodiments, the kit is assembled to form an implant where the stopper and cinching element abut against the helical anchor at opposed longitudinal ends thereof with the wire extending therebetween under tension.

In some embodiments, the kit further includes another helical anchor, where the kit is assembled to form an implant where the helical anchors are in prolongation of each other with a space therebetween and with the stopper and cinching element abutting against a respective one of the helical anchors opposed to the space with the wire extending therebetween under tension. In some embodiments, the anchor is rectilinear when undeformed and curved when the implant is assembled with the wire under tension. In some embodiments, the stopper is securable to the wire, the implant kit further including another stopper securable to the wire, another helical anchor, and another beaded wire.

In some embodiments, the kit is assembled to form an implant where the helical anchors are in prolongation of each other with a space therebetween with the stoppers abutting against a respective one of the helical anchors opposed to the space, each wire extending from a respective one of the stoppers through a respective helical anchor and reaching the cinching element provided adjacent the space, the wires both extending under tension through the gap.

Aspects of the disclosure herein provide an implant kit, including at least two helical anchors defining each an anchor passageway extending therethrough, a beaded wire defining longitudinally spaced apart beads, and a cinching element defining a one-way gap allowing passage of the wire therethrough in only one direction.

In some embodiments, the kit is assembled to form an implant where the wire forms a loop extending through both helical anchors and closed by the cinching element. In some embodiments, the loop forms an «8» figure. In some embodiments, in the implant, the two helical anchors are laterally spaced apart from each other with substantially parallel anchor passageways.

An implant assembled from the implant kit according to any one of the embodiments described above.

Aspects of the disclosure herein provide a wound closing device, including a cryoadhesion device including a hollow thermally conductive element and a cooling fluid supply for supplying a cooling fluid thereto, and an anchor driver for driving a helical anchor over the thermally conductive element. In some embodiments, the anchor driver supports the anchor at a distal end of a rigid member.

Aspects of the disclosure herein provide a method of cutting a wire using a transcatheter system, the method including advancing a transcatheter system including a catheter and a cutting member having a distal annular blade through at least one bodily vessel of a subject to a position at or near a target tissue, driving one or more anchors into the target tissue using a driving catheter, cinching a wire extending through the one or more anchors, and cutting at least a portion of the wire using the cutting member. In some embodiments, the annular blade is configured to both translate and rotate relative to the wire. In some embodiments, the method further includes cryoadhering at least one of the one or more anchors to a portion of the tissue. In some embodiments, the method further includes biasing the annular blade towards the wire.

Advantageously, the proposed system and various components thereof are usable to perform many different types of surgery efficiently.

Other objects, advantages and features of the present disclosure will become more apparent upon reading of the following non-restrictive description of some embodiments thereof, given by way of example only with reference to the accompanying drawings.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIG. 1, in a schematic view, illustrates a transcatheter surgery system in accordance with an embodiment of the present disclosure;

FIG. 2, in a perspective view, illustrates a cooling catheter part of the system of FIG. 1 in accordance with an embodiment of the present disclosure;

FIGS. 3, 4, 5, 6, 7, 8A, 8B, 9, 10, 11, 12, 13, 14, 15, 16, 17, 18, 19, 20, and 21, in side elevation and perspective views, illustrate various aspect of the cooling catheter of FIG. 1 and of some variants thereof in accordance with embodiments of the present disclosure;

FIG. 22, in a side elevation view, illustrates an actuator usable with the cooling catheter of FIG. 2 and a driving catheter illustrated in FIG. 23;

FIG. 23, in a side elevation view, illustrates a driving catheter part of the system of FIG. 1, here shown slid over the cooling catheter of FIG. 1;

FIGS. 24, 25, 26, 27A, 27B, and 28, in various views, illustrate various aspects of the driving catheter of FIG. 23 and the actuator of FIG. 22;

FIG. 29, in a longitudinal cross-sectional view, illustrates a cinching catheter part of the system of FIG. 1;

FIGS. 30, 31, 32, 33, 34, 35, 36, 37, 38, 39 and 40, in various views, illustrate various aspect of the cinching catheter of FIG. 29 and of some variants thereof;

FIG. 41, in a perspective cut away view, illustrates an actuator usable with the cinching catheter of FIG. 29 in accordance with an embodiment of the present disclosure;

FIG. 42, in a perspective view, illustrates the actuator of FIG. 40;

FIG. 43, in an exploded view, illustrates a cutting catheter part of the system of FIG. 1 in accordance with an embodiment of the present disclosure;

FIGS. 44, 45, 46, and 47, in various views, illustrate various aspect of the cutting catheter of FIG. 43;

FIGS. 48A, 48B, and 48C, in a flowchart, illustrate an annuloplasty procedure using the system of FIG. 1 in accordance with an embodiment of the present disclosure;

FIG. 49, in a flowchart, illustrates an alternative annuloplasty procedure using the system of FIG. 1 in accordance with an embodiment of the present disclosure;

FIG. 50, in a flowchart, illustrates another alternative annuloplasty procedure using the system of FIG. 1 in accordance with an embodiment of the present disclosure;

FIG. 50bis, in a flowchart, illustrates yet another alternative annuloplasty procedure using the system of FIG. 1 in accordance with an embodiment of the present disclosure;

FIG. 51, in a flowchart, illustrates yet another alternative annuloplasty procedure using the system of FIG. 1 in accordance with an embodiment of the present disclosure;

FIG. 52, in a flowchart, illustrates an AP reduction procedure using the system of FIG. 1 in accordance with an embodiment of the present disclosure;

FIG. 53, in a flowchart, illustrates an alternative AP reduction procedure using the system of FIG. 1 in accordance with an embodiment of the present disclosure;

FIGS. 54A and 54B, in a flowchart, illustrate a suturing procedure using the system of FIG. 1 in accordance with an embodiment of the present disclosure;

FIG. 55, in a flowchart, illustrates an edge-to-edge valve repair procedure using the system of FIG. 1 in accordance with an embodiment of the present disclosure;

FIGS. 56, 57, 58, 59, 60, 61, 62, 63, 64, 65, 66, 67, 68, 69, 70, 71, 72, 73 and 74, in schematic views, illustrates various steps of the method of FIGS. 48A to 48C in accordance with embodiments of the present disclosure;

FIGS. 75, 76, 77, and 78, in schematic views, illustrates various steps of the method of FIG. 49 in accordance with embodiments of the present disclosure;

FIGS. 79, 80, 81, and 82, in schematic views, illustrates various steps of the method of FIG. 50 in accordance with embodiments of the present disclosure;

FIGS. 83 and 84, in schematic views, illustrates various steps of the method of FIG. 50Bis in accordance with embodiments of the present disclosure;

FIGS. 85, 86, and 87, in schematic views, illustrates various steps of the method of FIG. 51 in accordance with embodiments of the present disclosure;

FIGS. 88 and 89, in schematic views, illustrates various steps of the method of FIG. 52 in accordance with embodiments of the present disclosure;

FIGS. 90 and 91, in schematic views, illustrates various steps of the method of FIG. 53 in accordance with embodiments of the present disclosure;

FIGS. 92, 93, 94, 95, 96, 97, 98, 99, 100, 101, 102, 103 and 104, in schematic views, illustrates various steps of the method of FIGS. 54A and 54B and variants thereof;

FIGS. 105 and 106, in schematic views, illustrates various steps of the method of FIG. 55 and variants thereof;

FIG. 107, in a perspective view, illustrates an alternative actuator usable in the system of FIG. 1 in accordance with embodiments of the present disclosure;

FIG. 108, in a side elevation view, illustrates a cooling supply actuator in a first configuration;

FIG. 109, in a side elevation view, illustrates the cooling supply actuator in a second configuration in which the cooling fluid supply has been moved relative to the first configuration;

FIG. 110, in a schematic view, illustrates a manner of joining two sides of a wound for suturing;

FIG. 111, in a schematic view, illustrates another manner of joining two sides of a wound for suturing;

FIG. 112, in a perspective view, illustrates an alternative driving catheter;

FIG. 113, in a perspective exploded view, illustrates an alternative cutting catheter actuator usable with an alternative cutting catheter in accordance with embodiments of the present disclosure;

FIG. 114, in a perspective view, illustrates a knob part of the cutting catheter assembly of FIG. 113;

FIG. 115, in a perspective view, illustrates a distal extremity of a support member part of the cutting catheter usable with the cutting catheter actuator of FIG. 113;

FIG. 116, in a side partially cut away view, illustrates a cutting catheter including the cutting catheter assembly of FIG. 113 in a first configuration;

FIG. 117, in a side elevation view, illustrates detail CXVII of FIG. 116;

FIG. 118, in a side partially cut away view, illustrates the cutting catheter of FIG. 116 in a second configuration;

FIG. 119, in a side elevation view, illustrates detail CXIX of FIG. 118;

FIG. 120, in a side partially cut away view, illustrates the cutting catheter of FIG. 116 in a third configuration;

FIG. 121, in a side elevation view, illustrates detail CXXI of FIG. 120; and

FIG. 122 illustrates the cinching wire formed in a loop passing through one or more anchors in accordance with an embodiment of the present disclosure.

### DETAILED DESCRIPTION

The terms “substantially” and “about” are used throughout this document to indicate variations in the thus qualified terms. These variations are variations that do not materially affect the manner in which the invention works and can be due, for example, to uncertainty in manufacturing processes or to small deviations from a nominal value or ideal shape that do not cause significant changes to the invention. Also, the terminology “proximal” and “distal” refers to a position relative to an operator using the present disclosure on a patient. Distal elements are closer to an intervention site, in the patient, and proximal elements are closer to the operator, for example a surgeon, using the proposed invention.

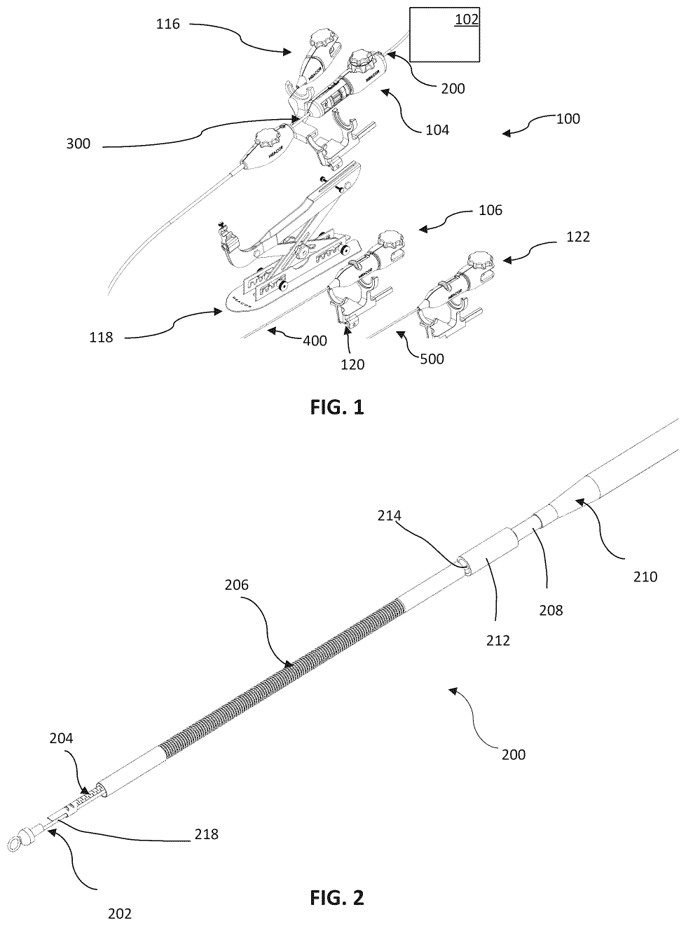

Referring to FIG. 1, there shown schematically a transcatheter surgery system 100, simply referred to hereinbelow as the system 100. The system 100 may include a cooling catheter 200, a driving catheter 300, a cinching catheter 400 and a cutting catheter 500 and wire tensioning device 600. The cooling, driving, cinching and cutting catheters 200, 300, 400 and 500, respectively, may be usable to perform various transcatheter procedures or other minimally invasive surgeries, a few examples of which are described below. The system 100 also may include a cooling system 102 for providing a cooling fluid, a cooling and driving catheter actuator 104 for moving various components of the cooling catheter 200, and a cinching catheter actuator 106 for operating the cinching catheter 400. In some embodiments, a cutting catheter actuator 122 like the cinching catheter actuator 106, or a similar actuator, may be also usable to actuate the cutting catheter 500. For example, the cooling fluid may be a pressurized gas that is configured to expand at a suitable lower pressure location in the cooling catheter 200 to cool a portion thereof. The cooling system 102 therefore may include any component required to provide the pressurized gas in a controlled manner. However, in alternative embodiments, any other suitable cooling fluid may be provided, such as for example compressed or liquefied gas canisters or a mixed-phase gas and liquid. Stands 118 and 120 may be used to support the various actuators used in the system 100.

Referring to FIG. 2, the cooling catheter 200 may include a pulling assembly 202, a wire guide 204, a bellow 206, an inner tube 208, an outer tube 210, a sleeve 212 and a cooling fluid supply 214. The pulling assembly 202 and the wire guide 204 and usable to selectively curve the cooling catheter 200 to allow steering through a patient's vasculature and setting the shape of the bellow 206 so that the latter can cool a tissue adjacent thereto and eventually adhere to this tissue through cryoadhesion. The cooling fluid supply 214 receives the cooling fluid from the cooling system 102 (not shown in FIG. 2) to cool the bellow 206. In the drawings, only a distal end of the cooling catheter 200 is illustrated, with understanding that the cooling catheter 200 may be long enough to be inserted through a conventional surgical catheter, or sheath, to perform surgery at a site of interest, such as inside the heart. The various components of the cooling catheter 200 that require access from the outside to perform the surgery therefore have a sufficient length to provide this access. In some embodiments, the diameter of the bellow 206 may be adjusted using a pull-wire.

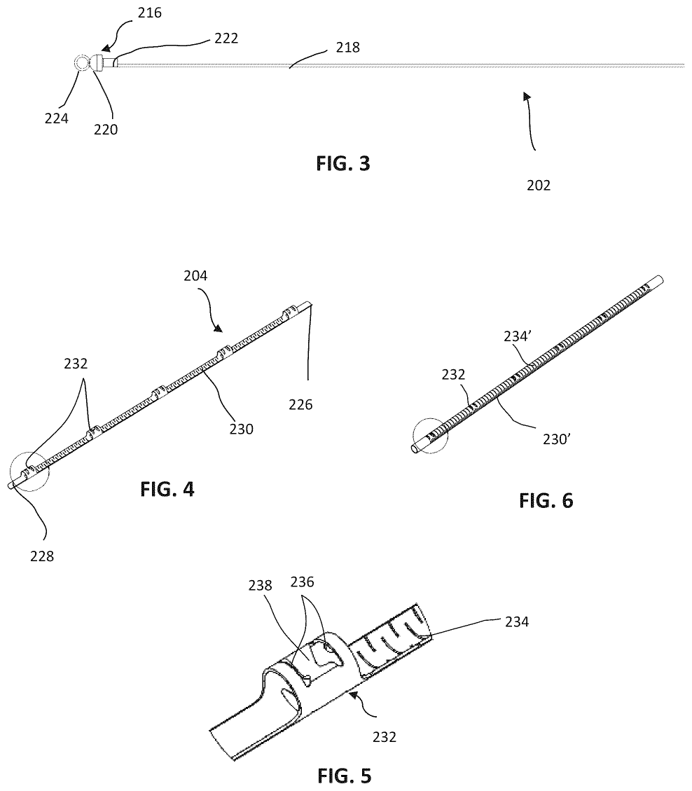

FIG. 3 illustrates the pulling assembly 202. The pulling assembly 202 may include an end cap 216 and a pull wire 218 extending therefrom. The end cap 216 may include a dome 220 having a substantially atraumatic shape, such as a hemispherical shape, and a cap sleeve 222 extending therefrom. In some embodiments, an external wire attachment 224 extends from the dome 220 opposed to the pull wire 218 to be outside of the cooling catheter 200 when the latter is in use. The wire attachment 224 may be usable to attach an external wire thereto that may be used for example for cinching in valvular annuloplasty. In a specific embodiment, the wire attachment 224 takes the form of a ring.

FIG. 4 illustrates the wire guide 204. The wire guide 204 may be elongated and typically manufactured by removing suitable portions of a tubular member. The wire guide 204 defines longitudinally opposed wire guide proximal and distal ends 226 and 228. In a first embodiments, the wire guide 204 may include a longitudinally extending backbone 230 from which a plurality of longitudinally spaced apart cylindrical tubes 232 extend. The backbone 230 defines a plurality of notches 234 (better seen for example in FIG. 5) extending transversally partway therethrough and longitudinally spaced apart from each other. The notches 234 facilitate bending of the backbone 230 in a plane perpendicular to the notches 234. The tubes 232 receive the pull wire 218 therethrough when the wire guide 204 and the pulling assembly 202 may be assembled.

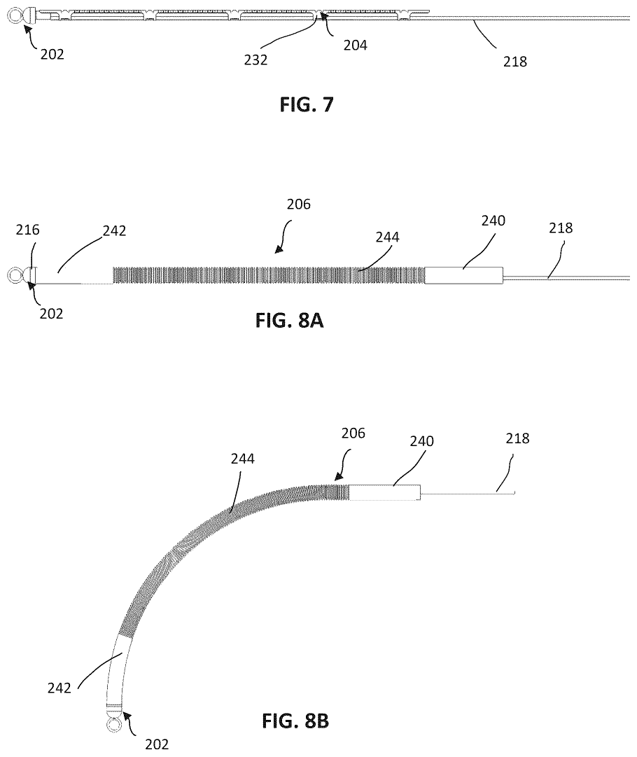

In some embodiments, during manufacturing, a pair of slits 236 may be formed in each of the tubes 232 opposed to the backbone 230. The slits 236 each extend circumferentially around part of the tube 232 and together define a tab 238 therebetween. As seen in FIG. 6, the tabs 238 can be pushed inwardly inside the tubes during manufacturing. This allows insertion of the pull wire 218 between the outer wall of the tubes 232 and the inwardly pushed tabs 238 to provide a relatively narrow passageway through which the pull wire 218 may extend, as seen in FIG. 7. This maintains the pull wire 218 spaced apart from the backbone 230, which creates a uniform pull force along the tip replicating a multi-lumen tube thereabout to facilitate bending of the cooling catheter and maintain bend plane using the pull wire 218. Typically, the pull wire 218 may be slidable relatively easily relative to the tubes 232 while remaining relatively close to the periphery of the tube 232. Thus, when the pull wire 218 is pulled, the wire guide 204 is configured to bend in a plane perpendicular to the backbone 230 and notches 234, or in other words in a plane extending both through the backbone 230 and the tabs 238.

Once assembly is completed, the backbone 230 and the sleeve 222 overlap longitudinally. This transmits efficiently to the remainder of the cooling catheter 200 the force and torques created when the pull wire 218 is pulled. In the embodiment shown in the drawings, releasing the pull wire 218 results in the backbone 230 resuming its rectilinear undeformed shape passively. In alternative embodiments, a two-wire, or a multi-wire (3 or 4 wires for example), antagonist system may be used to perform this deformation instead.

In alternative embodiments, as seen in FIG. 6, the backbone 230′ may be tubular and provided with slits 234′ configured to allow bending of the backbone 230′ in a predetermined plane.

FIGS. 8A and 8B illustrate the bellow 206. The bellow 206 is configured to relatively easily transfer heat, so that when the interior of the bellow 206 is cooled by the cooling fluid, any tissue against which the bellow 206 abuts is relatively quickly cooled. Eventually, this tissue will be cooled below freezing and the bellow 206 and the tissues will adhere to each other. For example, the bellow 206 is formed from a relatively thin metal tube of a sufficient thickness to withstand a pressurized cooling fluid.

The bellow 206 is a thermally transmissive portion that is configured to adhere to tissue once the bellow 206 is cooled internally. The bellow 206 may include longitudinally opposed bellow proximal and distal end sections 240 and 242 and a bellow intermediate section 244 extending therebetween. The bellow proximal and distal end sections 240 and 242 may be more rigid than the bellow intermediate section 244, the latter defining convolutions allowing the bellow intermediate section 244 to bend when the backbone 230 is deformed by pulling on the pull wire 218. In some embodiments, the bellow 206 may include a braided tubing that is flexible enough to withstand high pressures. In some embodiments, the bellow 206 is multi-lumen. In some embodiments, the cooling catheter may be built to withstand relatively high internal pressures, for example up to 3000 psi.

When the cooling catheter 200 is assembled, the bellow 206 may be positioned around the wire guide 204, which itself receives the pull wire 218, and the bellow 206 abuts against the cap 216. The cap 216 may be typically fixedly secured to the bellow distal end section 242, for example through soldering. The bellow intermediate section 244 and the backbone 230 at least partially overlap so that they can jointly deform to deform the cooling catheter 200 from an unstressed configuration, as seen in FIG. 8A, to a curved configuration, seen in FIG. 8B. For example, in the curved configuration, the bellow intermediate section 244 may be substantially arcuate. In some embodiments, the bellow 206 may be relatively long, for example long enough to extend along a major portion of a cardiac valve annulus or other tissue.

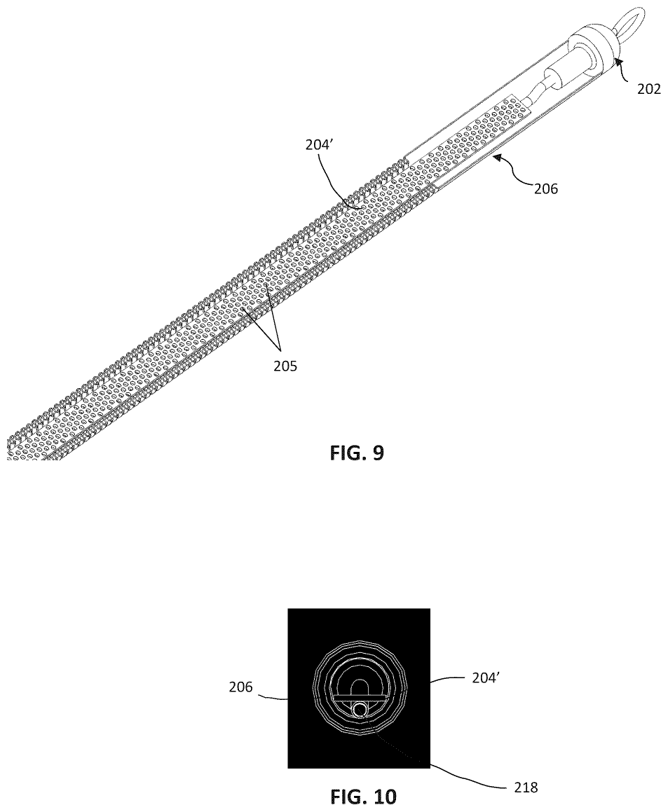

In alternative embodiments, as seen in collectively FIGS. 9 and 10, the wire guide 204′ takes the form of a perforated plate fixed inside the bellow 206. As seen in FIG. 10, the pull wire 218 may be provided between the pull wire and the bellow 206, a gap between the pull wire 218 and the bellow 206 being of a dimension suitable to relatively snugly receive the pull wire 218 while allowing the latter to slide slightly relative thereto. The perforated plate bends predominantly in a plane perpendicular thereto and include perforations 205 that extend therethrough allow the cooling fluid to fill the bellow 206, while also facilitating bending of the wire guide 204′.

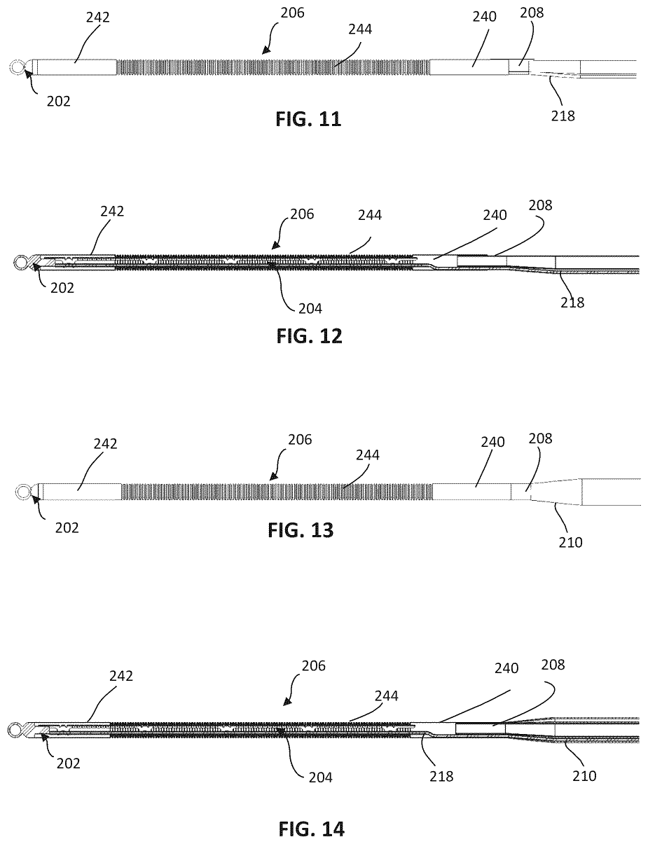

Referring to FIGS. 11 and 12, an inner tube 208 may be fitted inside the bellow proximal end section 240, shy of the wire guide 204. The pull wire 218 exits the bellow 206 outside of the inner tube 208. The inner tube 208 may be mounted fixedly inside the bellow 206, for example using an adhesive, while allowing the pull wire 218 to more relative thereto.

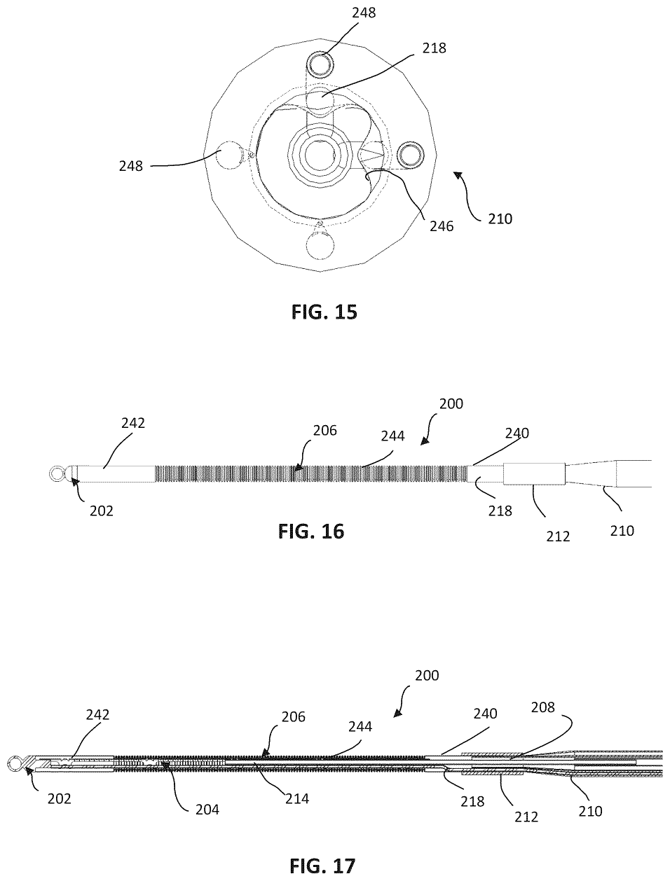

Referring to FIGS. 13 and 14, the inner tube 208 and pull wire 218 may be fitted inside the outer tube 210, all three components reaching outside of the patient in a typical transcatheter procedure. As seen in FIG. 15, the outer tube 210 may take the form of a multi-lumen catheter including a main lumen 246 through which the inner tube 208 and pull wire 218 extend, and auxiliary lumens 248 that through which accessories, such as a thermocouple, can be inserted. As seen in FIG. 16, in some embodiments, a relatively rigid sleeve 212 may be fitted over the bellow proximal end section 240 and the outer tube 210 (which tapers distally to allow such fitting).

Finally, referring to FIG. 17, the cooling fluid supply 214 may take the form of a tube extending from within the bellows to 206 to outside of the outer tube 210. The cooling fluid supply 214 may be for example configured to deliver a pressurized gas, for example through a relatively small opening at its distal tip, such that when the gas expands, the gas may be cooled, which in turn cools the bellows 206. The expanded gas then exits the bellow 206 through the outer tube 210. In some embodiments, the cooling fluid supply 214 may be fixed relative to the bellow 206. In other embodiments, the cooling fluid supply 214 may be movable longitudinally along the remainder of the cooling catheter 200. In some embodiments the cooling fluid supply 214 could have multiple openings or exits along its distal end spaced apart such as the cooling effect is spread over a distance. Once the cooling gas is flowing inside the catheter, the polymer, polyimide, or plastic tubing is configured to expand and compress against this stainless-steel sleeve, making it leak-proof.

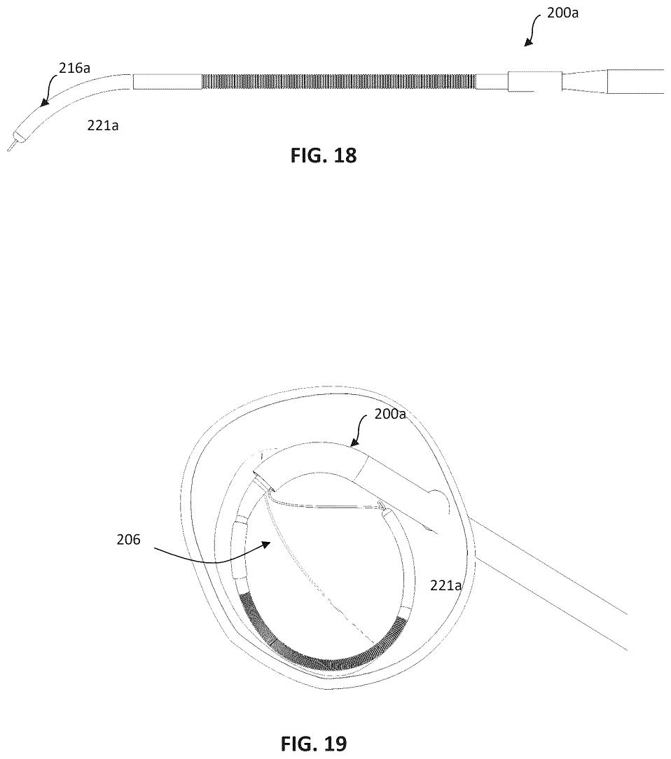

Referring to FIG. 18, in some embodiments, the cooling catheter 200a may include a section that may adopt a predetermined non-straight configuration when no forces are exerted thereonto. For example, the cap 216a may include a tubular section 221a of arcuate configuration or a D-shaped configuration, for example of shape and dimensions matching similar shape to the annulus section. This cap 216a may be shaped or pre-bent. In such embodiments, the tubular section 221a can help the user position the bellow 206 faster by guiding a surgeon to the right location with minimal trial and error, as seen in FIG. 19. In other embodiments, the tubular section 221 may be replaced by a preformed wire.

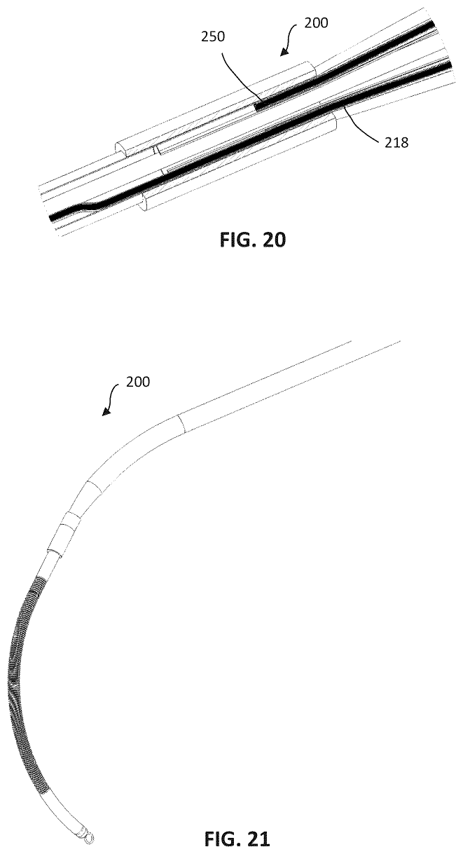

Referring to FIG. 20, in some embodiments, one or more auxiliary pull wires 250 may be fitted in one of the auxiliary lumen 248 of the outer tube 210 and secured to the outer tube 210 at its distal end. This auxiliary pull wire 250 may be for example at about 90 degrees about the outer tube 210 relative the pull wire 218 and may further help in positioning of the cooling catheter 200 by better controlling its deformation in various planes, as seen in FIG. 21. In other embodiments, some sections of the cooling catheter 200 may be pre-bent to assume predetermined shapes when no external forces are exerted thereonto to facilitate positioning and handling of the cooling catheter.

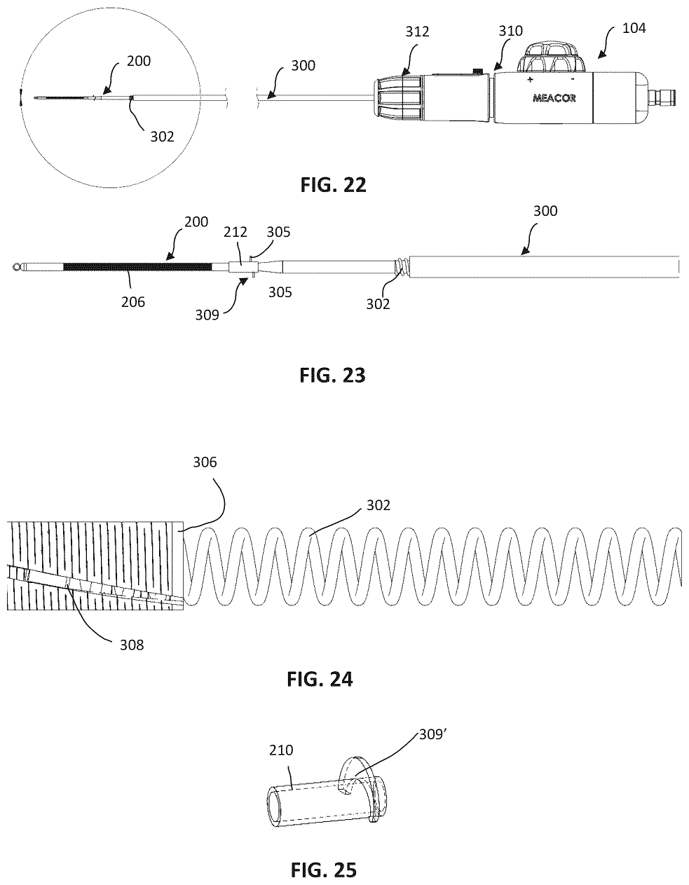

Referring to FIG. 22, there is shown the driving catheter 300. The driving catheter 300 may be usable with the cooling and driving catheter actuator 104 and one or more anchors 302. Referring to FIG. 23, the driving catheter 300 may be slidable over the cooling catheter 200 so that the anchor 302 may be inserted in tissue (not seen in FIG. 23) to which the bellow 206 may be adhered. This sliding movement may be also used to load the anchors 302 to the driving catheter. The driving catheter 300 may be also rotatable relative to the cooling catheter 200. The driving catheter 300 may be an elongated tube that is flexible enough to be used in transcatheter surgery, while still being able to transmit sufficient torque to drive the helicoidal anchor 302 into tissue.

As seen in FIG. 24, the anchor 302 may be secured a distal end 306 of the driving catheter 300. The anchor 302 may be typically removable from the driving catheter 300. For example, the anchor 302 takes the form of an elongated helicoidal member terminating in a point that may be screwed in a suitable thread 308 formed internally at the distal end 306 of the driving catheter 300. When the anchor 302 abuts against a tissue and the driving catheter 300 may be rotated in a driving direction, the anchor 302 is configured to be driven into the tissue. When the anchor 302 is anchored past the distal tip of the driving catheter 300, the anchor 302 is configured to be released and the driving catheter 300 can be released. The anchor 302 will be left behind as the driving catheter 300 is removed. Alternative anchors 302 and driving catheters 300 that may be usable in the present disclosure are described in PCT patent application PCT/IB2018/060073 filed Dec. 14, 2018, the contents of which is hereby incorporated by reference in its entirety.

Referring to FIG. 23, in some embodiments, the sleeve 212 may be small enough to fit inside the anchor 302 and is provided with an anchor engagement 309 configured for engaging the anchor 302 and ensuring that the anchor 302 is driven into the tissue according to its pitch. For example, the anchor engagement 309 takes the form of one, two or more pins 305 protruding radially outwardly from the sleeve 212 and longitudinally and angularly separated from each other to match the pitch of the helicoidal anchor 302. A seen in FIG. 25, in other embodiments, the engagement 309′ takes the form of a helical flange extending from the sleeve 212, once again with a pitch matching the pitch of the helical anchor 302. In addition, the engagements 309 and 309′ prevent the anchor 302 from releasing without rotation and prevent the driving tube 306 from extending beyond the location of the engagements 309 and 309′, so that this prevents elongation of the driving tube for better control.

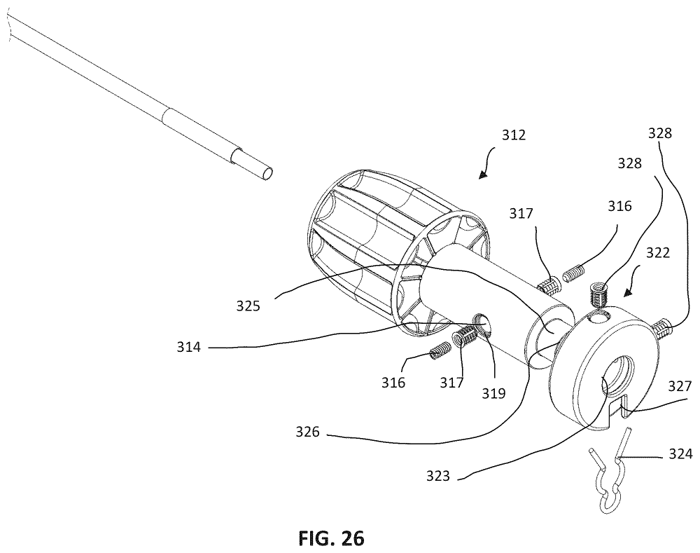

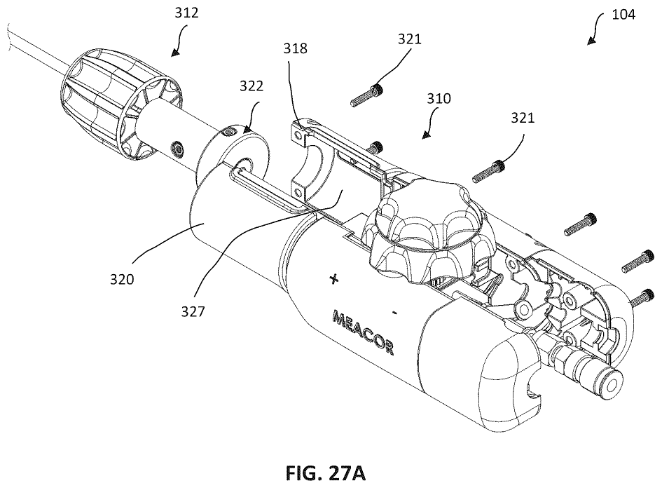

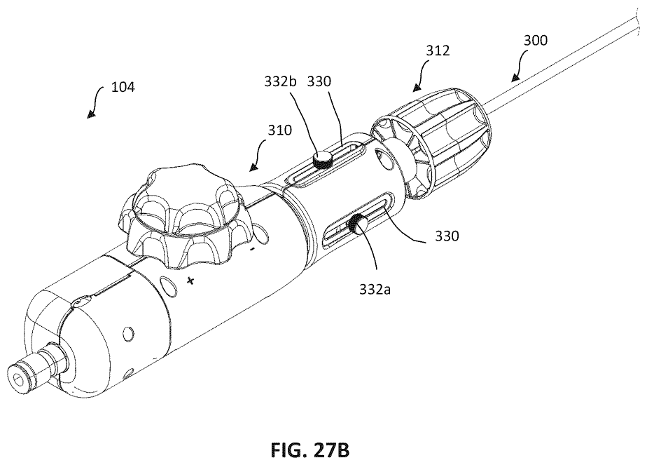

The actuator 104 may be used to rotate the driving catheter 300. For example, referring collectively to FIGS. 26 to 28, the actuator 104 may include a casing 310 to which a knob 312 defining an axial knob passageway 314 may be mounted. The driving catheter 300 extends through the knob passageway 314 and may be immobilized relative thereto using set screws 316 engaging tubular threaded inserts 317 fixedly mounted in suitable mounting apertures 319 formed in the knob 312 and extending radially therethrough.

In some embodiments, the knob 312 may be removable from the casing 310 of the actuator 104 to facilitate assembly of the actuator 104 with the cooling and driving catheters 200 and 300. For example, the casing 310 may include two parts 318 and 320 that each extend the whole length of the casing 310 and that secured to each other using screws 321, allowing one to split the casing 310 into two to allow access to its interior. A knob mount 322 to which the knob 312 may be mounted is removably mountable to the casing 310 when the two parts 318 and 320 may be separated from each other. The knob 312 can rotate freely relative to the knob mount 322 unless locked thereto.

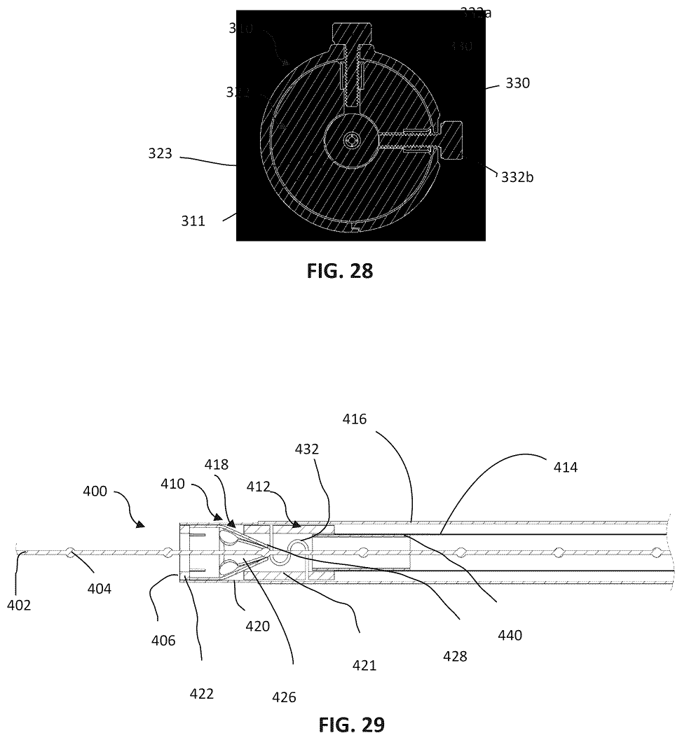

For example, the knob mount 322 may be substantially annular and defines a central aperture 323 through which a proximal mounting shaft 325 part of the knob 312 may be mounted. A cotter pin 324 can be used to engage a circumferential groove 326 formed in the mounting shaft 325. The cotter pin 324 may be inserted in a suitable slit 327 formed in the knob mount 322 and reaching the central aperture 323. In some embodiments, the knob mount 322 may be provided with one or more threaded inserts 328 mounted thereto and opening radially outwardly relative thereto. The casing 310 may be provided with a pair of longitudinally extending elongated slits 330 positioned such that when the knob mount 322 is suitably rotated, screws 332a and 332b can be used to engage the threaded inserts 328 to immobilize the knob mount 322 relative to the casing 310. (See FIG. 28) When mounted to the casing 310, the knob mount 322 may be received in a cylindrical cavity 327 formed inside the casing 310 to be rotatable and slidable there along.

The screws 332a and 332b engage the knob mount 322 through the slits 330, thereby locking rotations of the knob mount 322 relative to the casing 310. In an aspect, one of the screws 332a may be too short to reach the mounting shaft 325, so that when this screw 332a is fully screwed, the knob mount 322 and the casing 310 are prevented from moving longitudinally relative to one another through frictional engagement, as the casing 310 is then sandwiched and pressed between the screw 332a and the knob mount 322. In an aspect, the other screw 332b may be long enough to protrude in the central aperture 323 when fully screwed, to then engage the mounting shaft 325 to prevent rotation of the latter relative to the knob mount 322, and therefore to the casing 310. Therefore, each of the rotation and translation of the knob 312 relative to the casing can be individually locked, which allows one to do the same with the driving catheter 300, as the latter may be fixedly mounted to the knob 312.

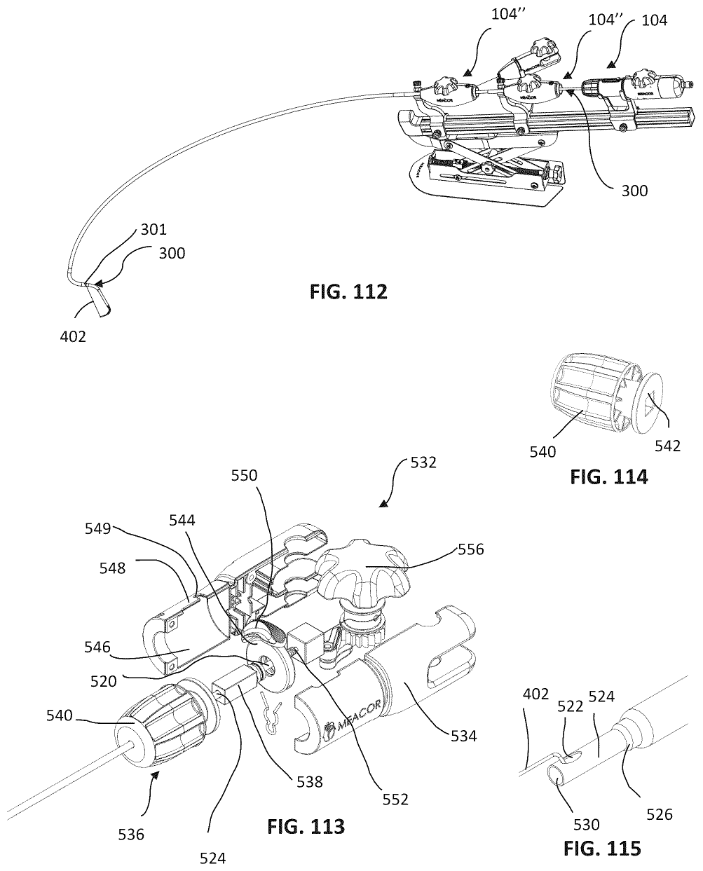

As seen in FIG. 112, in some embodiments, the cinching catheter 300 may include a double shell construction including two layers axially rotatable relative to each other and configured for receiving a cinching wire 402, further described below, therebetween to prevent wire entanglement while anchor driving. This double shell construction is achieved by adding an outer driving catheter sheath 301 outside the driving catheter 300. The outer driving catheter sheath 301 may be steerable using an auxiliary steering catheter actuator 104″, and many steerable concentric sheaths independently steerable and movable relative to each other may be added to enhance steerability.

The cinching catheter 400 may be usable to tighten a loop of wire that is inserted in a patient. For example, as detailed below, valvular annuloplasty can be performed by anchoring two or more helicoidal anchors 302 around the annulus of the valve with a wire inserted through the anchors 302. When the wire is cinched, the diameter of the annulus may be reduced, thereby reducing the dimensions of the aperture that the valve leaflet has to cover. Such a cinching action can also be performed in many other surgical procedures.

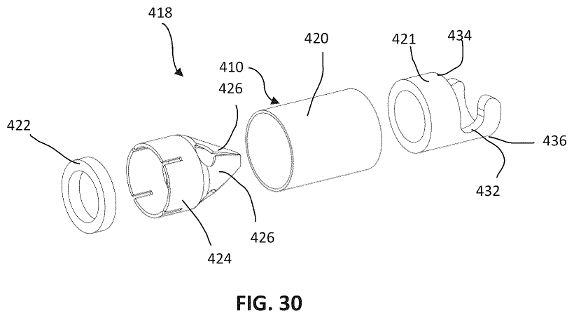

Referring collectively to FIGS. 29 and 30, the cinching catheter 400 may be usable with a cinching wire 402. The cinching wire 402 typically may include a plurality of beads 404 at longitudinally spaced apart locations there along. The beads 404 may be formed, for example and non-limitingly, with knots tied in the cinching wire 402. The cinching wire 402 extends proximally from outside the cinching catheter 400 and exits from the cinching catheter 400 at a cinching catheter distal end 406 thereof. The cinching wire 402 may be anchored distally inside the patient (to tissue or to an implant) or may loop back to outside the patient outside of the cinching catheter 400.

The cinching catheter 400 may include a cinching element 410, a cinching element holder 412, an elongated tube 414 and a cinching element lock 416. The cinching element 410 may be left behind after the cinching procedure is completed, to maintain the cinching wire 402 relatively taut inside the patient, typically through mechanical interference with implants, such as anchors 302, or in some embodiment with tissue. Typically, the cinching element 410 also acts as a ratchet to allow the cinching wire 402 to be unidirectionally tightened at regular intervals, determined by the spacing of the beads 404. To that effect, the cinching element 410 may include a stopper 418 that allows the beads 404 to move proximally therethrough but prevents the beads 404 from moving distally therethrough. The cinching element holder 412 may be secured to the elongated tube 414 and cinching element 410 when the cinching element lock 416 is in a locked configuration. When the cinching element lock 416 is moved to an unlocked configuration, the cinching element 410 may be released and the remainder of the cinching catheter 400 can be removed, leaving behind the cinching element 410.

More specifically, the cinching element 410 may include a tubular body 420 terminated distally by an annular cap 422 fixed thereto. The stopper 418 may be mounted in the tubular body 420 and abuts against the cap 422. The stopper 418 may be provided between an attachment 421 secured to the tubular body 420 and the cap 422. The attachment 421 may be configured to mechanically interfere with the cinching element holder 412 to secure the cinching element 410 to the remainder of the cinching catheter 400.

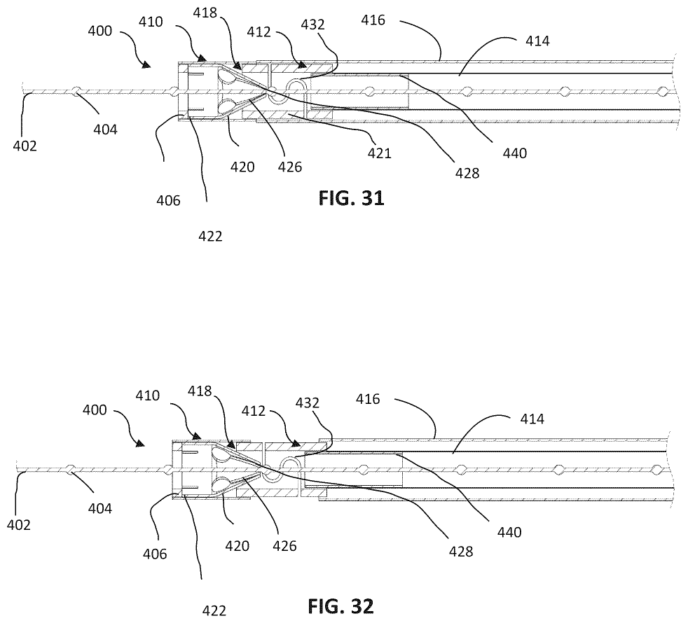

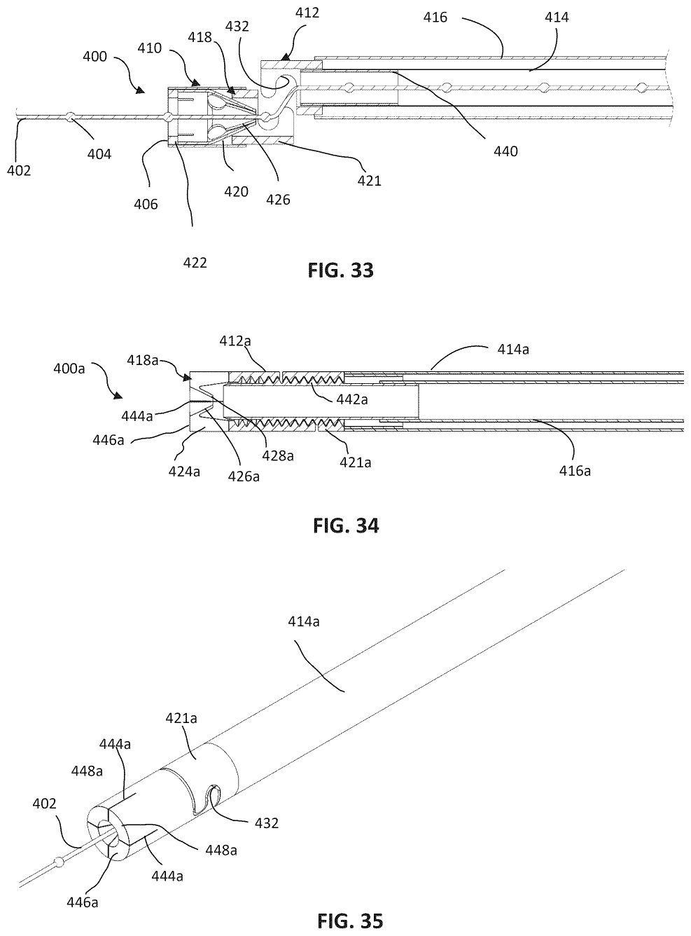

The stopper 418 may include for example a tubular stopper body 424 fitting snugly inside the tubular body 420 from which leaves 426 extend proximally. For example, four leaves are provided, but any other suitable number of leaves 426 may be used. The leaves 426 taper in a direction leading proximally and define a proximally located gap 428 therebetween. The cinching wire 402 extends through the gap 428. The gap 428 may be large enough to allow the cinching wire 402 to move longitudinally there along, but small enough to prevent the beads 404 to move in a distal direction therethrough. The leaves 426 may be deformable independently of each other so that the gap 428 may be enlarged when the beads 404 are guided by the leaves 426 towards and through the gap 428. For example, the leaves 426 may be separated from each other with cuts made at the tip to create the gap 428 and longitudinally there along to allow the leaves 426 to move relative to each other and separate from each other when suitable forces are exerted thereonto. Thus, in this embodiment, the leaves 426 extend integrally as a single piece of material with the stopper body 424 may be hinged at the stopper body 424 through a living hinge so that they can spread apart from each other through resilient deformations. The beads 404 engage the leaves 426 through their inner surfaces when the cinching wire 402 is pulled proximally and the beads approach the gap 428, which pushes the leaves 426 away from each other from a narrow configuration (seen for example in FIG. 31) to a wide configuration (seen for example in FIG. 29) to allow passage of the beads 404. Once the bead 404 has passed through the gap 428, the leaves 426 resiliently spring back towards each other, so that pulling the cinching wire distally relative thereto will cause the beads to abut against the tip of the leaves 426 and stop, as seen in FIG. 31. In some embodiments, the leaves 426 may be made of a Ni—Ti alloy showing shape memory effects and super elasticity, commercialized under the name Nitinol™.

The attachment 421 and the cinching element holder 412 may be complementarily shaped. For example, the attachment 421 and the cinching element holder 412 are formed by cutting a tubular member along an interface curve 432. The interface curve 432 has a shape such that the attachment 421 and the cinching element holder 412 may be only movable relative to each other transversally. If such transversal movements are prevented, longitudinal and rotational movements of the attachment 421 and the cinching element holder 412 relative to each other are impossible. For example, the interface curve 432 starts from an apex 434 to terminate at a nadir 436. The interface curve 432 follows a longitudinally extending S-shape along two laterally opposed sides of the attachment 421 and cinching element holder 412 assembly, following the contour of the tube from which this assembly is manufactured. The S-shape monotonically goes from the apex 434, at a distalmost location thereof to the nadir 436, at a proximal most location thereof, so that no transversal plane intersects the S-shape at spaced apart locations there along. This allows free transversal movements of the attachment 421 and the cinching element holder 412 relative to each other when they are unlocked. The S-shape prevents longitudinal movements as there are some longitudinal lines along the attachment 421 and the cinching element holder 412 assembly that intersect the S-shape curve at longitudinally spaced apart locations there along, which creates mechanical interference. Finally, since the S-shape may be formed by cutting a cylinder, rotations are prevented as the S-shape does not show a rotation symmetry that would allow rotations. In some embodiments, the attachment 421 and the cinching element holder 412 are substantially like each other, so that they look identical when one is flipped relative to the other one.

The cinching element tube 414 may be long enough to protrude outside the patient when a transcatheter procedure may be performed. The cinching element holder 412 may be mounted distally to the cinching element tube 414, for example through a tubular coupler 440 extending therebetween and secured thereto. The cinching element holder 412 and attachment assembly 421 has an outer diameter substantially like that of the cinching element tube 414

The cinching element lock 416 takes the form of an elongated tube surrounding the cinching element tube 414 and reaching the cinching element 410. In a locked configuration, as seen in FIG. 29, the cinching element lock is moved so that the attachment 421 is engaged thereinto. This prevents the cinching element 410 from detaching from the cinching element tube 414 as detailed above. When the cinching element lock 416 is moved proximally until the attachment 421 may be entirely outside of the cinching element lock 416, as seen in FIG. 32, the cinching element 410 is released and the remainder of the cinching catheter 400 can be withdrawn with the cinching element 410 remaining behind, mounted to the wire 402.

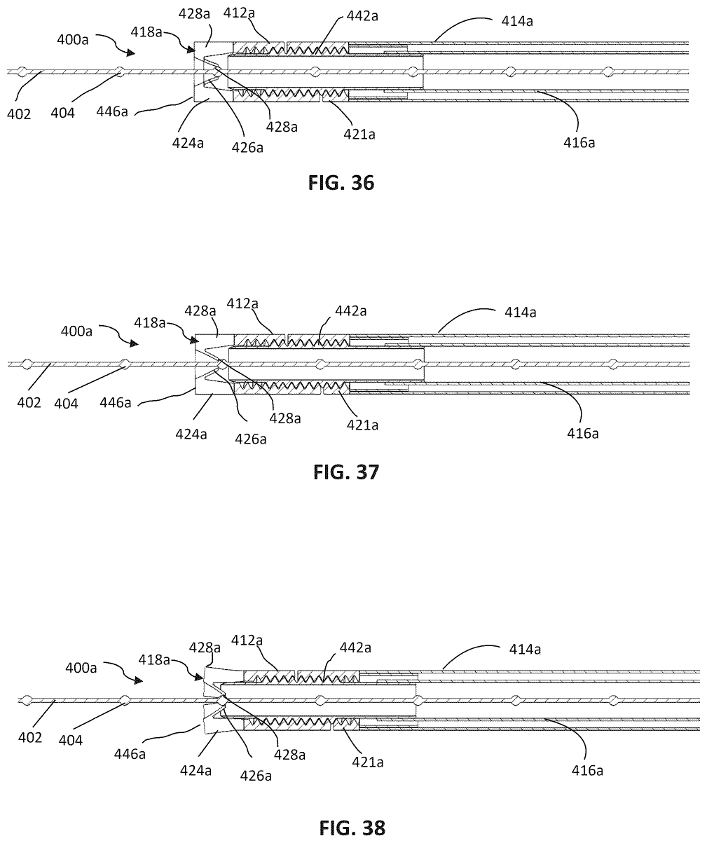

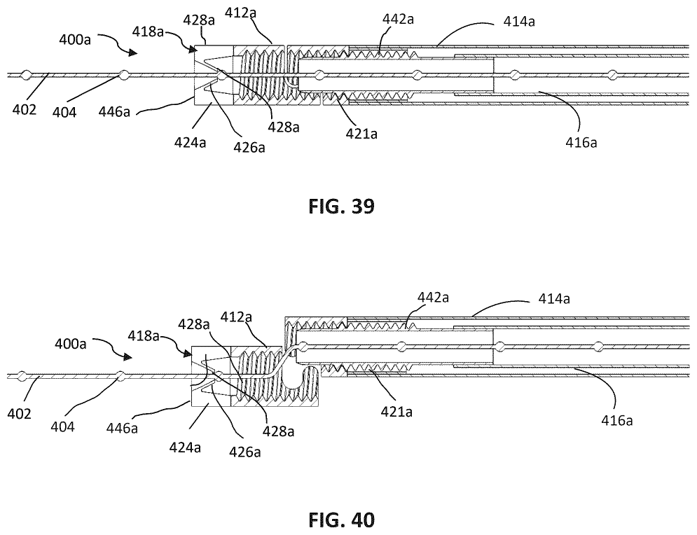

FIGS. 34 to 40 illustrate an alternative cinching catheter 400a. In this cinching catheter 400a, the cinching element lock 416a takes the form of a tube extending through the interior of the elongated tube 414a and including a distally located externally threaded section 442a, and the attachment 421a and the cinching element holder 412a may be internally threaded, so that the cinching element lock 416a locks the attachment 421a and the cinching element holder 412a to each other when the threaded section 442a engages them both. Once the cinching element lock 416a in unscrewed from the attachment 421a, as seen in FIGS. 39 and 40, the latter becomes detached from the cinching element holder 412a.

In some embodiments, the stopper 418a may include a tubular body 424a in which the leaves 426a extend internally, forming a conical shape tapering proximally, as described above. Slits 444a may be formed in the tubular body 424a and extend longitudinally partially there along from its distal end 446a. The slits 444a thus define tubular body sectors 448a each supporting a respective leaf 426a. The slits 444a therefore extend proximally further than the leaves 446a. The internal surface of the tubular body 424a may be curved so that a diameter of the tubular body 424a tapers in a direction leading distally. The tubular body sectors 448a may be configured such that they are resiliently deformable outwardly and the cinching catheter 400a may be adapted to selectively open the gap between the leaves 426a to allow both distally and proximally oriented movements of the beaded wire 402 therethrough. To that effect, when the cinching element lock 416 may be screwed in so that it protrudes inside the tubular body 424a and engages the tubular body sections 448a, the latter may be deformed such that the gap 428a between the leaves 446a may be enlarged. If a sufficient deformation is allowed, one can thus enlarge the gap 428a enough to allow the beads 404 to move distally, as well as proximally through the gap 428a, as seen in FIG. 38. This allows one to correct overtightening of the cinching wire 402 if such overtightening occurs.

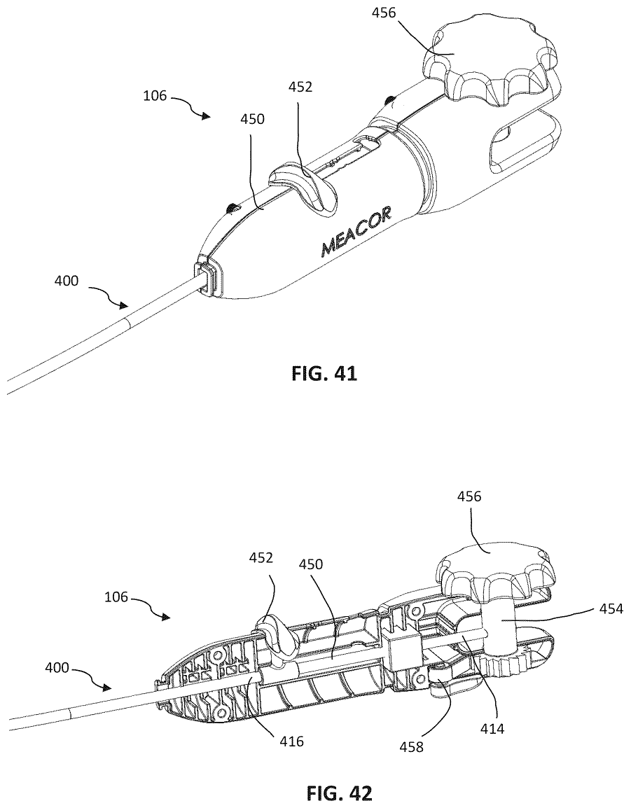

FIGS. 41 and 42 illustrate a cinching catheter actuator 106 usable for operating the cinching catheter 400. The cinching catheter actuator 106 may include a casing 450 to which may be mounted a slider 452 and a spool 454. The slider 452 may be movable longitudinally along the casing 450. In some embodiments, notches or other suitable features may be provided to be engaged by the slider 452 so that the latter is not too easily moved along the casing 450. Other manners of preventing the slider 452 from moving inadvertently are also possible. The spool 454 may be located inside the casing 450 and may be rotatable by an external knob 456. In some embodiments, a releasable ratchet mechanism 458 may be also provided that only allows movements of the spool 454 in a predetermined direction unless released.

The cinching element lock 416 may be mounted to the slider 452 to be jointly movable therewith along the casing 450. The elongated tube 414 may be mounted to the casing 450 to be fixed relative thereto. The cinching wire 402 (not shown in FIGS. 41 and 42) may be wound around the spool 454. Therefore, moving the slider 452 relative to the casing 450 moves the cinching element lock 416 between the locked and unlocked configurations, while rotating the knob 456 allows one to pull the cinching wire 402 inside the casing 450. Releasing the ratchet mechanism 458 allows one to remove some cinching wire 402 from the spool 454. The ratchet mechanism can be used in addition to or in replacement of the beads 404 and may be omitted in some embodiments.

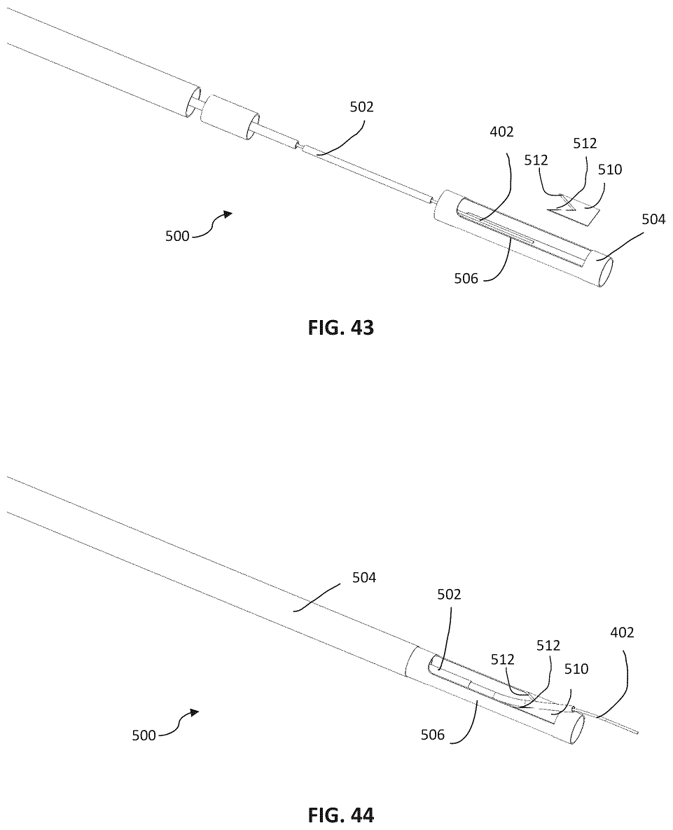

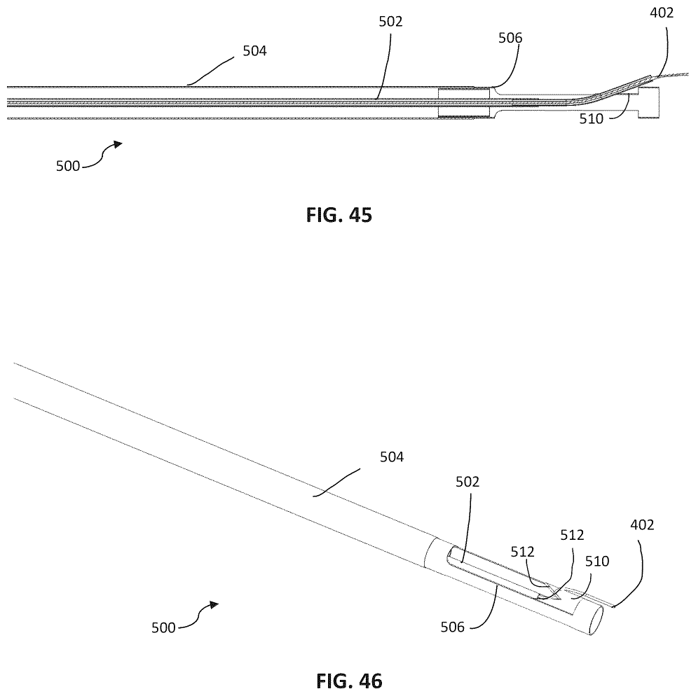

Referring to FIGS. 43 and 44, the cutting catheter 500, which may be called sometimes a release catheter, may be usable to cut the cinching wire 402 or any other wire, such as a suture, remotely, inside the patient from the outside. The cutting catheter 500 may include cutting catheter inner and outer tubes 502 and 504 that are longitudinally movable relative to each other. Each of the cutting catheter inner and outer tubes 502 and 504 may be manufactured in two parts joined to each other through a tubular coupler, to allow each replacement of worn-out parts, or to manufacture relatively short parts out of metals, while the main part of the inner and outer tubes 502 and 504 may be made of flexible polymers. The inner tube 502 may be typically relatively small in diameter as compared to the outer tube 504.

The outer tube 504 may be provided with a laterally extending outer tube aperture 506 shy of the outer tube distal end 508. The outer tube aperture 506 for example has a substantially rectangular configuration and spans about 45 to 180 degrees around the outer tube 504. In some embodiments, two diametrically opposed outer tube apertures 506 may be provided. A V-shaped blade 510 may be mounted to the outer tube 504 in the outer tube aperture 506, the V-shape blade 510 tapering in a distally oriented direction and including a pair of distally converging and proximally facing cutting edges 512.

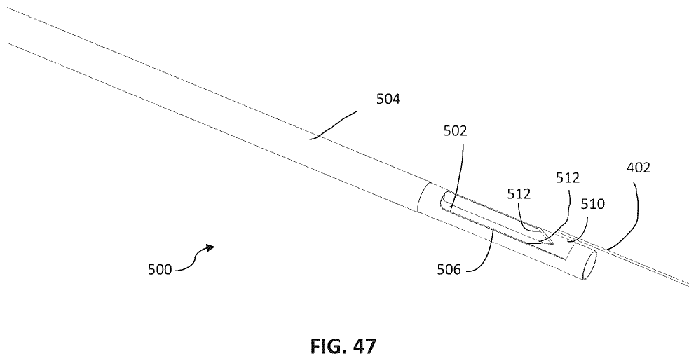

The cutting catheter 500 may be usable as follows. Initially, as seen in FIG. 44, the inner tube 502 may be positioned to protrude from the outer tube aperture 506, with the wire 402 that will be eventually cut extending through the inner tube 502. To cut the wire 514, the inner tube 502 may be withdrawn inside the outer tube 504, which results in the wire 402 abutting against the blade 510, as seen in FIG. 46. The wire 402 may be maintained under slight tension, and the inner tube 502 may be then pushed. The inner tube 502 may be rigid enough to continue being pushed past the outer tube aperture 506 inside the outer tube 504, which creates a fold in the wire 402. This fold abuts against the cutting edges 512, and with a suitable tension in the wire 402 coupled with a sufficient force exerted on the inner tube 502, the wire 402 may be cut by the blade 510, as seen in FIG. 47, and the cutting catheter 500 can be removed from the patient. The cutting catheter actuator 122 may be like the cinching catheter actuator 106, except that the slider 452 may be coupled to the inner tube 502 to slide the latter along the outer tube 502. In some embodiments, cutting and cinching catheters like the components described in this document may be integrated in a single cutting/cinching device, with their actuators integrated in a single handle.

Referring collectively to FIGS. 113 to 121, there is shown an alternative cutting catheter 500′ for cutting a wire 402, such as a cinching wire, a suture, or any other similar suitable structure. The cutting catheter 500′ may include a cutting catheter actuator 532, or actuating assembly, to which may be mounted an elongated wire receiving member 520 provided with a wire receiver 522 at a wire receiving member distal end 523 thereof for receiving the wire 402. An elongated cutting member 524 provided with an annular blade 526 at a cutting member distal end 528 thereof may be mounted over the wire receiving member 520 so that the annular blade 526 may be positionable adjacent the wire receiver 522. The cutting member 524 may be longitudinally movable relative to the wire receiving member 520 and axially rotatable relative thereto so that the blade 526 can both translate and rotate relative to the wire 402 with the latter received in the wire receiver 522.

In some embodiments, the wire receiving and cutting members 520 and 524 may be both flexible and may be concentric, the cutting member 524 being tubular and surrounding at least part of the wire receiving member 520. In such embodiments, the wire receiving and cutting members 520 and 524 may be bendable to be able follow a patient's vasculature to perform a transcatheter procedure. It should be noted that performing cutting operations through a transcatheter procedure is notoriously difficult, as the flexibility required to position the cutting catheter adjacent a suture inside a patient complicates the cutting operation, as the required forces may be not well transmitted from outside the patient to the cutting site due to the flexibility of the device.

Advantageously, the proposed cutting catheter 500 solves this problem by combining rotation of the cutting member 524 relative to the wire holder 522 with having a cutting member 524 that is biased in a distally oriented direction relative to the wire receiver 522. Therefore, rotational forces, which may be easier to transmit using the flexible cutting member 524 than longitudinal forces, may be used to cut the wire 402 using rotation of the cutting member 524, while not requiring a large longitudinal force. Typically, the cutting member 524 turns over at least one full rotation before the wire 402 may be cut, and in some embodiments about 10 to 15 rotations may be required. Having the ability to rotate the cutting member 524 over numerous complete rotations helps in reducing the longitudinal force that must be exerted thereby to achieve a successful cutting action. Typically, longitudinal movements may be axial rotations of the cutting member 524 relative to the wire receiving member 520 may be independent from each other.

The wire receiving member 520 may be any suitable element able to immobilize the wire 402 relative to the cutting member 524. For example, the wire receiving member 520 may be elongated and hollow, with the wire receiver 522 taking the form of a wire aperture extending transversally through the wire receiving member at its distal end. The wire receiving member 520 defines a wire receiving member passageway 530 extending longitudinally therethrough and opening in the wire aperture.

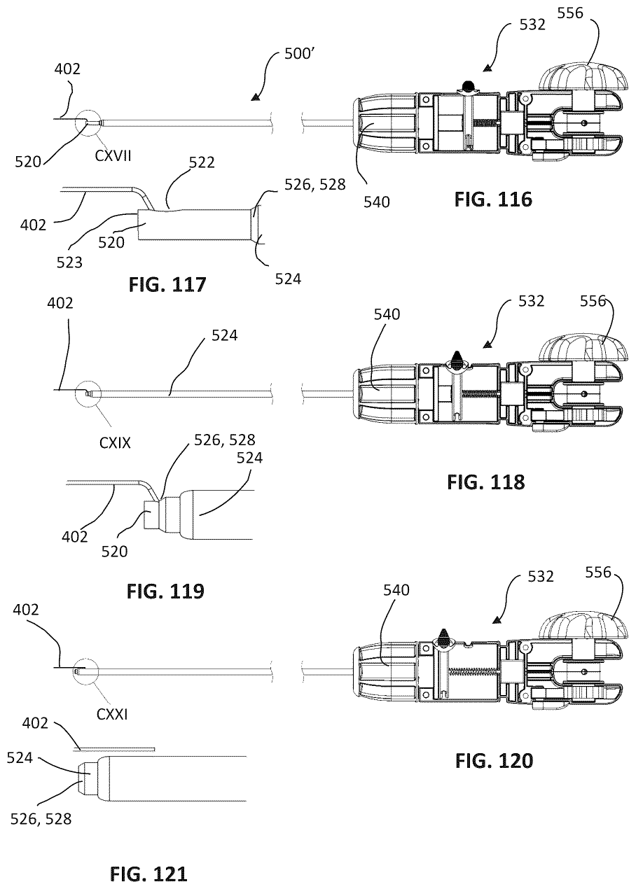

The cutting catheter actuator 532 may include a body 534, the wire receiving member 520 being mounted to the body 534. The cutting catheter actuator 532 also may include a cutting member actuator 536 operatively coupled to the cutting member 524 for selectively and independently rotating and longitudinally moving the cutting member 524 relative to the body 534. The cutting member 524 may be movable between proximal and distal positions relative to the body 534, as seen respectively in FIGS. 116 and 120. In some embodiments, the cutting member 524 may be lockable in translation relative to the body 534 in the proximal position, so that no accidental cutting action may occur while the cutting catheter 500′ is advanced inside the patient.

More specifically, the cutting member actuator may include a cutting member mount 538 and a knob 540. The cutting member 524 extends through the cutting member mount 538 and may be jointly axially rotatable therewith, for example by being glued thereto or integrally formed therewith, among other possibilities. The knob 540 may be mounted to the body 534 to be axially rotatable relative thereto and defines a knob aperture 542 extending axially therethrough receiving the cutting member mount 538 thereinto so that the cutting member mount 538 may be axially movable along the knob 540 and jointly rotatable therewith. This may be achieved in the actuating assembly 532 by having a cutting member mount 538 and knob aperture 542 of similar configurations and dimensions, for example having a substantially square transversal cross-sectional configuration.

The cutting member mount 538 may be mounted itself to a mounting element 544 to be able to rotate relative thereto, the mounting element 544 being movable longitudinally along a suitably shaped cavity 546 extending along the body 534. The body 534 also defined a slit 548 extending into the cavity 546 so that a slider 550 secured to the mounting element 544 through the slit 548 allows an intended user to move the mounting element 544 along the cavity 546 to position the cutting member manually. The slit 548 extends generally longitudinally, with a proximal notch 549 allowing positioning of the slider 550 thereinto to lock the cutting member 534 in the proximal position by rotating the slider into the notch 549. A biasing element 552 may be provided between the cutting member 524 and the body 534 for biasing the cutting member 524 towards the distal position. For example, the biasing element 552 may be a coil spring abutting against the mounting element 544 and biased against an internal structure inside the body 534 so that the biasing element 552 pushes the cutting member 534 distally. A tensioner 556, like the knob 446 described hereinabove, may also be provided for applying a tension to the wire 402.

In operation, the slider 550 may be initially in the notch 552, and the cutting member may be in the proximal position, as seen in FIGS. 116 and 117. The wire 402 extends from the tensioner 556 into the wire receiving member 520 and exits through the wire holder 522. The wire 402 may be attached to other structures or to tissue such that when the tensioner 556 may be used to pull on the wire 402, the latter will be relatively taut.

Once one wishes to start the cutting procedure, the slider 550 may be moved outside of the notch 548 so that the slider 550, and the mounting element 544 may be pushed distally, which moves the cutting member 524 distally until the blade 526 abuts against the wire 402. Therefore, the wire 402 may be pinched between the blade 526 and a distal end of the aperture 522. This configuration may be seen in FIGS. 118 and 119. In this configuration, the knob 540 can be rotated axially, which is configured to cut progressively through the wire 402. Once the wire 402 is cut, the cutting member 424 may move to its distal position, as seen in FIGS. 120 and 121, and the slider 550 can be used to retract the blade 526 so that the cutting catheter 500′ can be removed from the patient.

The cooling catheter 200, driving catheter 300, cinching catheter 400 and cutting catheter 500 can be used in a variety of surgical procedures, a few of examples of which are detailed below.

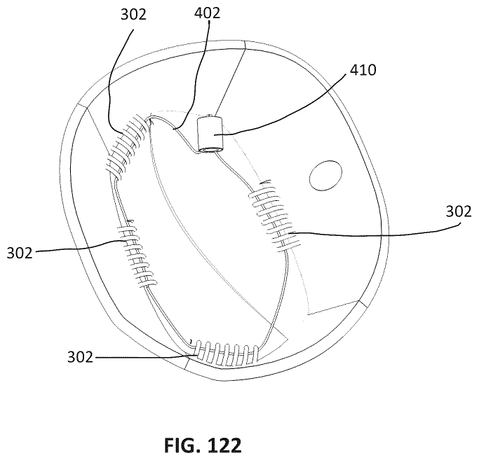

In yet other embodiments, as seen for example in FIG. 122, the cinching wire 402 may form a loop passing through one or more anchors 302, with both ends thereof entering a single cinching element 410, so that the stopper 613 is not required. In the embodiment of FIG. 122, four anchors 302 are shown.

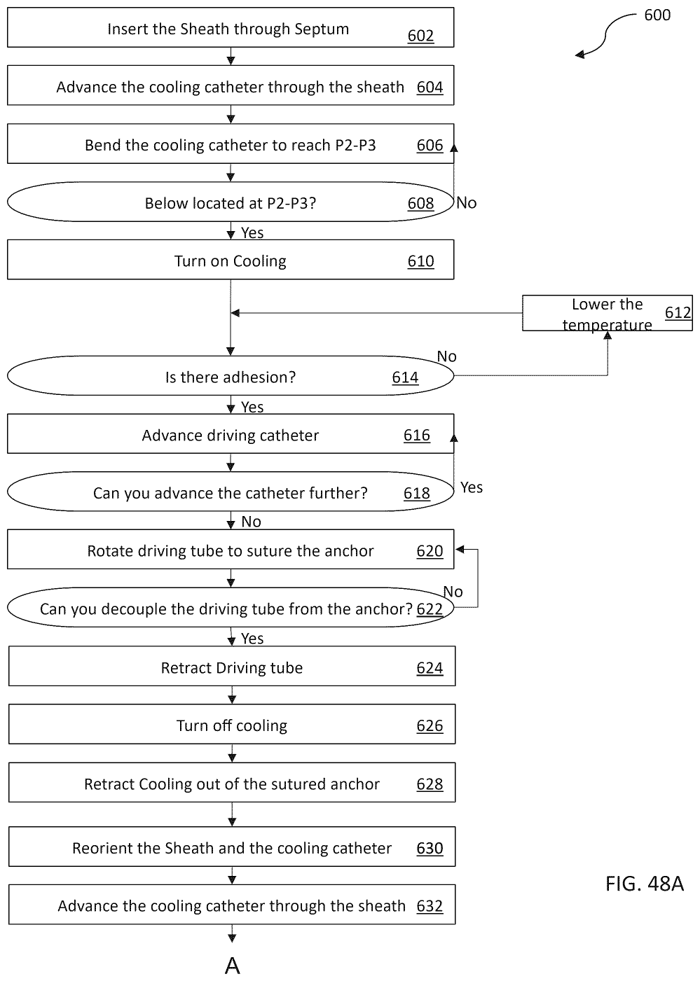

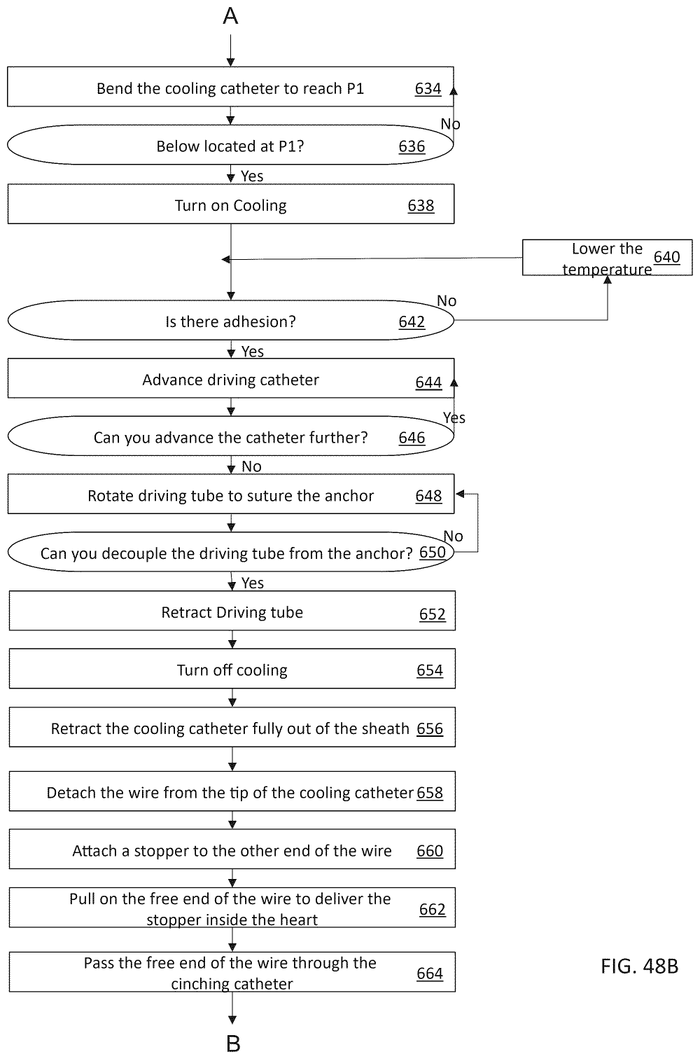

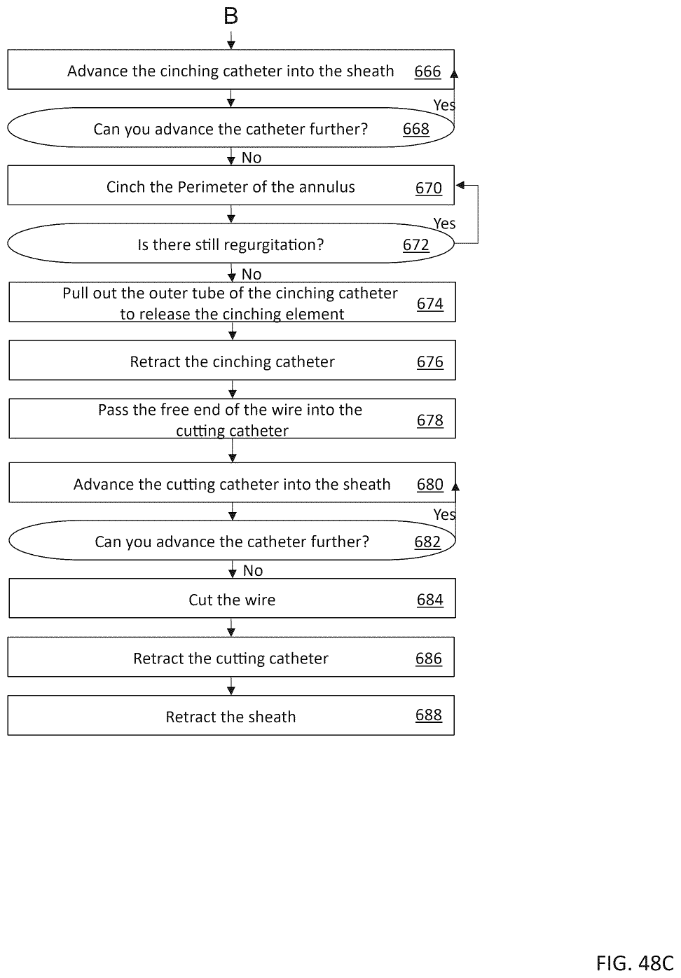

Referring to FIGS. 48A to 48C, in a first example, a method 600 of performing a valve repair procedure may be performed, and more specifically a mitral valve 609 repair. A similar procedure may be used to perform a tricuspid valve repair.

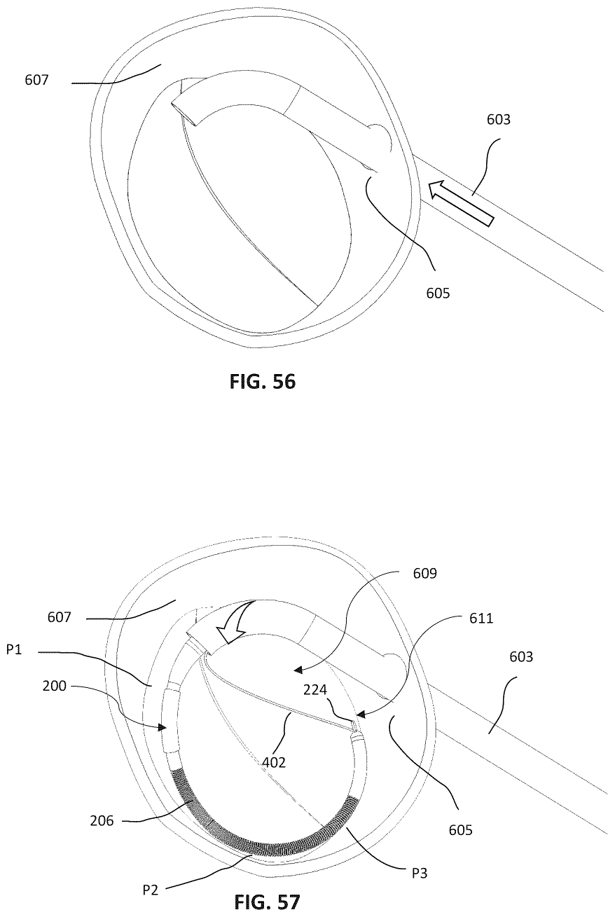

First, a sheath 603 may be inserted in a conventional manner in the patient's vasculature and driven through the septum 605 to reach the left atrium 607 (step 602) as seen in FIG. 56. Then, the cooling catheter 200 may be driven through the sheath 603 (step 604) and may be bent in a way to position the bellow 206 along various portions of the annulus 611 of the mitral valve 609 to insert anchors 302 around the annulus 611, followed by cinching.

More specifically, the cinching wire 402 may be attached to the wire attachment 224 and the cooling catheter 200 is first inserted through the sheath 603 and advanced and deformed until the bellow 206 is adjacent the P2 and P3 scallops (steps 606 and 608), and the cooling fluid is then delivered through the cooling fluid supply 214 (step 610). The resulting configuration may be illustrated in FIG. 57. Afterwards, the tissue may be cooled until the bellow 206 adheres to the surrounding tissues (steps 612 and 614) through cryoadhesion. This can be tested for example by trying to slightly move the cooling catheter 200. When there is adhesion, there will be no significant movements under small forces. Other anchoring sites around the mitral valve may be also usable in alternative embodiments.

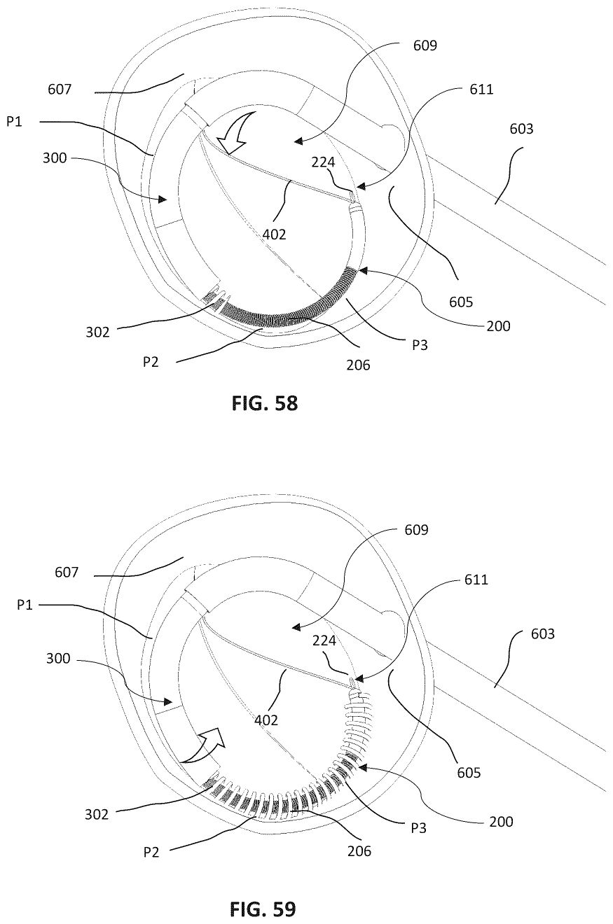

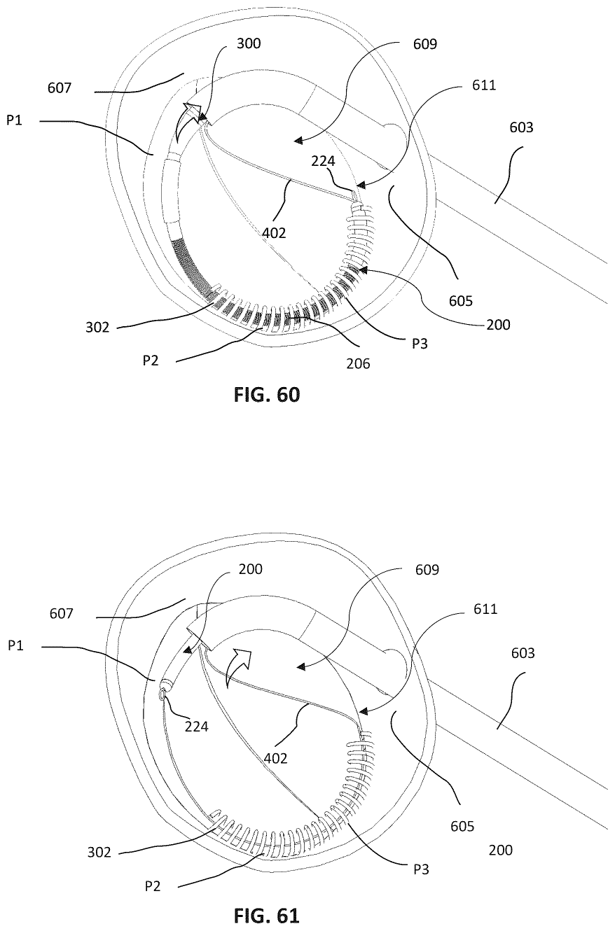

One anchor 302 may be then delivered through the sheath 603. To that effect, the driving catheter 300 to which an anchor 302 has been distally secured may be slid over the cooling catheter 200 until it cannot be further advanced as the anchor reaches the cryoadhesion site (steps 616 and 618), as seen in FIG. 58. The driving catheter 300 may be then rotated to suture the anchor 302 the annulus 311 using a helicoidal corkscrew motion. This may be performed until the anchor 302 can be detached, in the configuration shown in FIG. 59, by rotating the driving catheter 300 in a direction opposite the one used to drive in the anchor 302 (steps 618 and 620). Then, the anchor 320 may be driven fully inside the tissue, with the cooling catheter located in its central passageway. Thus, a longitudinally extending portion of the anchor 302 remains outside of the tissue, while a matching portion is inserted inside the tissue. Once the anchor 302 has been detached from the driving catheter 300, the driving catheter 300 may be retracted fully (step 624), as seen in FIG. 60, and cooling may be stopped (step 626). This causes detachment of the bellow 206 from the annulus. Then, the bellow 206 can then be retracted a bit until it is out of the anchor 302 (step 628), as illustrated in FIG. 61.

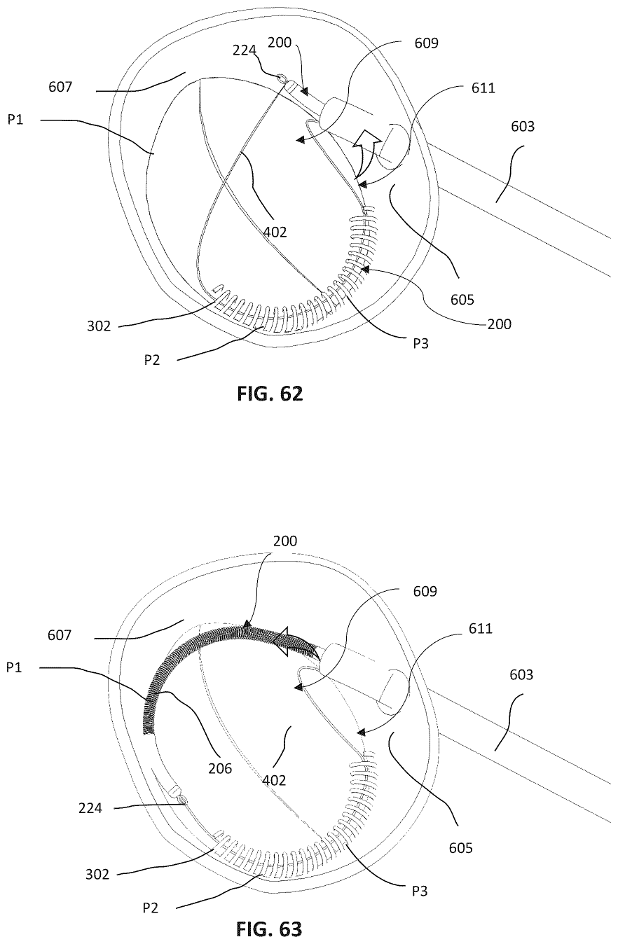

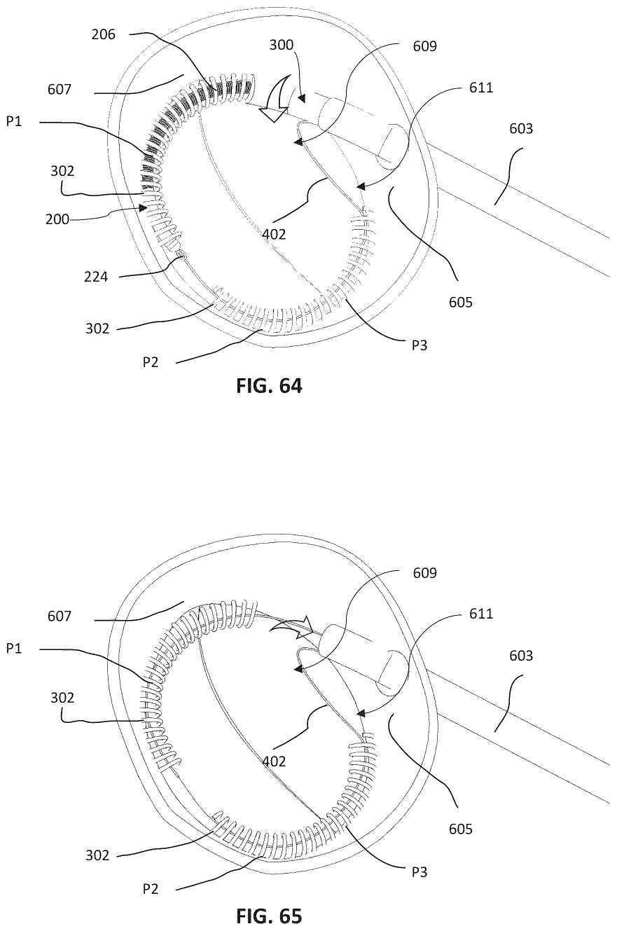

The sheath 603 can then be reoriented to get better access to another portion of the valve annulus 611, as seen in FIG. 62, and the same process can be repeated with the bellow 206 adjacent the P1 scallop to insert a second anchor 302 in a generally axial alignment with the first anchor 302 (steps 630 to 654), as illustrated in the sequence of FIGS. 63 and 64. Once the two anchors 302 are sutured, the cooling catheter 200 may be fully retracted from the sheath (step 656), as seen in FIG. 65 and the cinching wire 402 may be detached from the wire attachment 224 (step 658). The two anchors 302 may be very close to each other, abut against each other, or leave a gap therebetween. Note that once the cooling catheter 200 has been removed, the cinching wire 402 may be looped through the anchors 302, with two opposed ends of the cinching wire 402 reaching outside of the sheath 603.

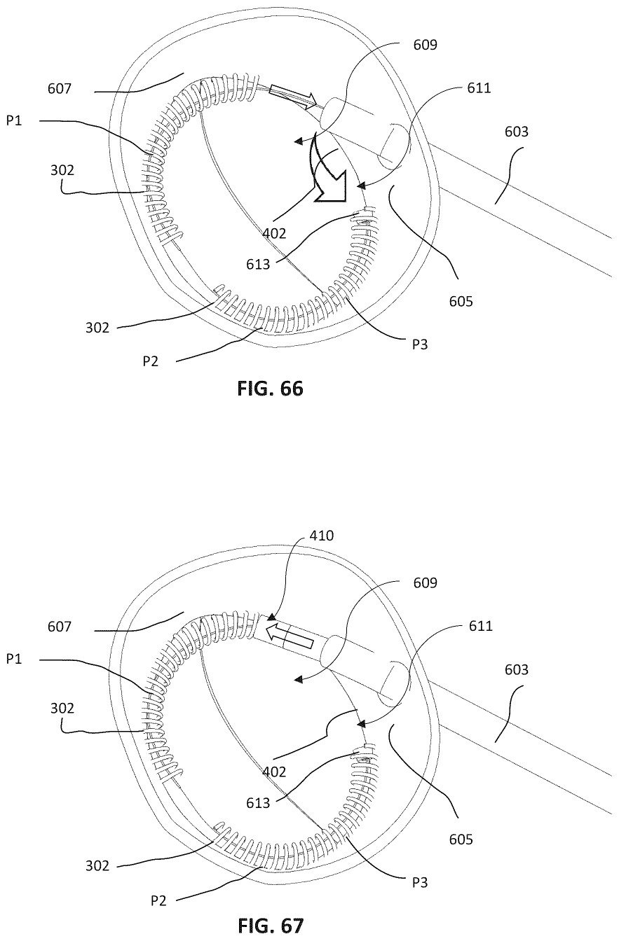

The cinching procedure can then be performed. Before cinching, one needs to deliver members that will abut against opposed ends of the two anchors 302. To that effect, a stopper 613 may be secured to one end of the cinching wire 402 (step 660). The stopper 613 may be a member that is larger than the diameter of the anchor but small enough to be delivered through the sheath 603. In some embodiments, the cinching wire 402 may be tied in a relatively large knot and the stopper 613 is therefore a section of the cinching wire. When the end of the cinching wire 402 opposed to the stopper 613 may be pulled, the latter is configured to travel through the sheath 603 until it abuts against one of the anchors 302, for example the anchor adjacent the P2-P3 site (step 662), as seen in FIG. 66. Then the portion of the cinching wire 402 that remains outside the sheath 603 can be inserted through the cinching catheter 400 (step 664), and the latter can be advanced through the sheath 603 until the cinching catheter cannot be advanced further (steps 664 to 668), as seen in FIG. 67. In this configuration, the cinching element 410 abuts against the anchor 302 opposed to the stopper 613.

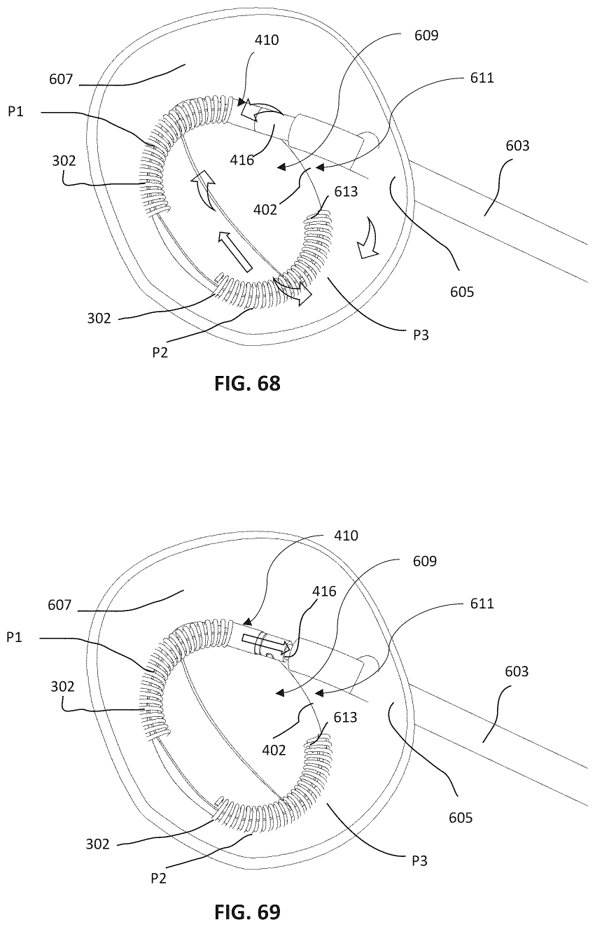

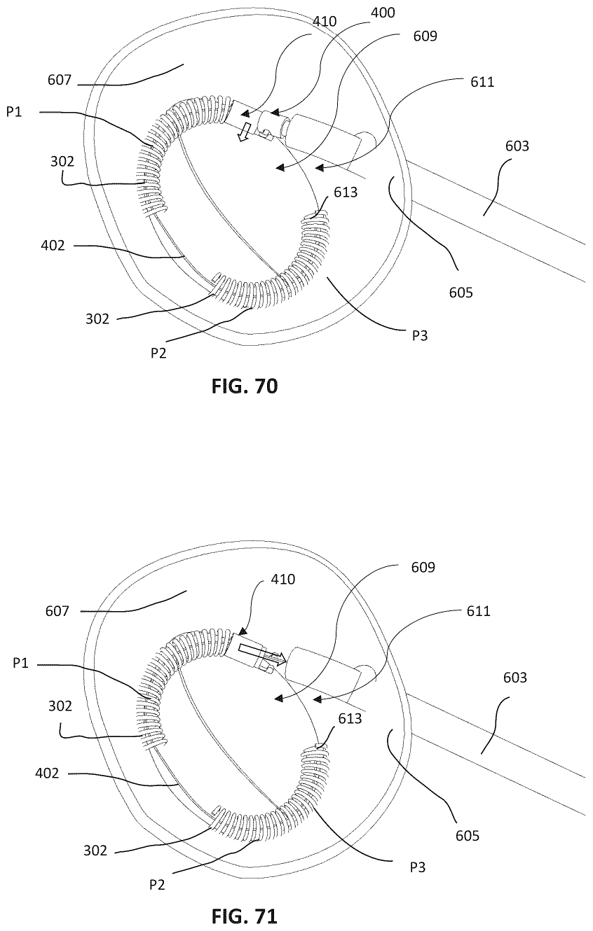

Cinching proper can then be performed. To the effect, the actuator 104 may be used to pull on the cinching wire 402 until there is reduced or no regurgitation (steps 670 and 672), as illustrated in FIG. 68. During this process, the stopper 613 and the cinching element 410 is configured to compress the two anchors 302 sandwiched therebetween, causing the tissue connected to them to shrink as well. This is configured to cause a decrease in the perimeter of the annulus 611, helping the leaflets of the mitral valve 609 to close properly, which stops, or at least reduces, regurgitation. Then, the cinching element lock 416 may be moved to its unlocked position to release the cinching element 410 (step 674), as seen in FIGS. 69 and 70, and the cinching catheter 400 may be removed from the sheath 603 (step 676), as seen in FIG. 71.

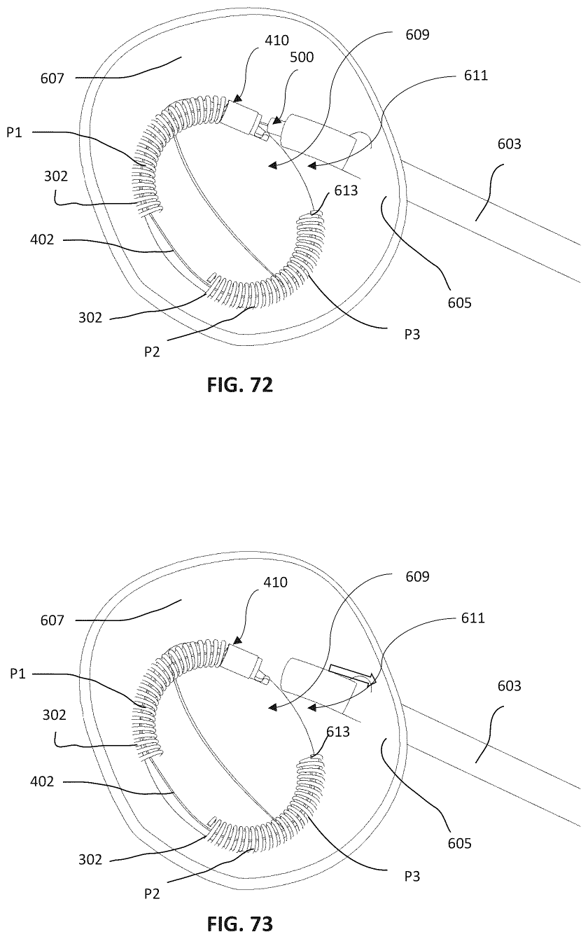

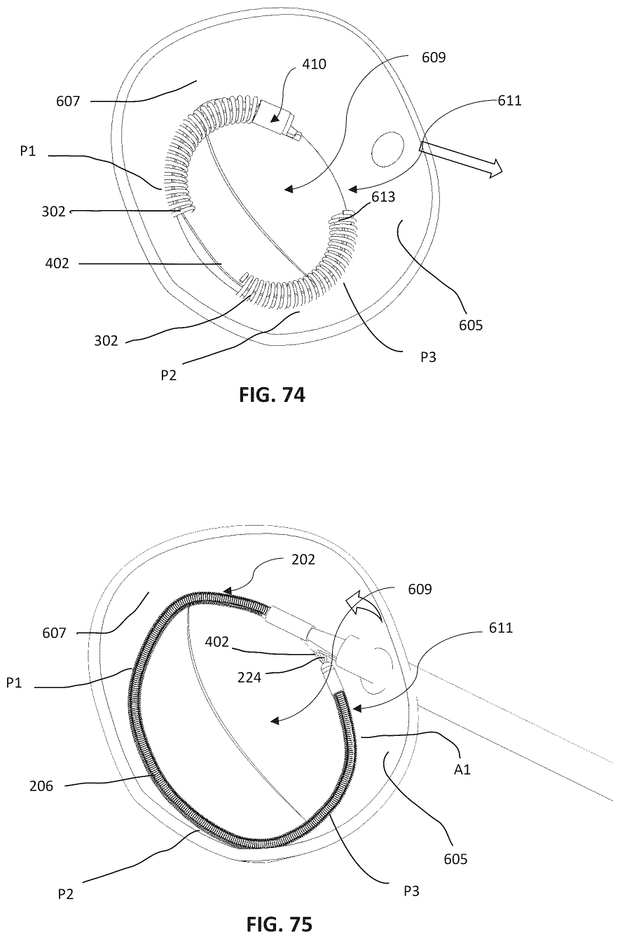

To complete the procedure, the cinching wire 402 may be cut at the cinching element 410. To that effect, the cinching wire 402 may be inserted through the cutting catheter 500 (step 678), and the latter can be advanced as far as possible towards the cinching element 410 (steps 680 and 682), as seen in FIG. 72. Then, the cutting catheter 500 may be used to cut the cinching wire 402, and described above (step 684), and the cutting catheter 500 and sheath 603 may be retracted (steps 686 and 688), as seen in the sequence of FIGS. 73 and 74. The cinching procedure may be now completed, and the cinching element 410, the stopper 613, the anchors 302 and a small section of cinching wire 402 extending through the anchors 302 between the stopper 613 and the cinching element 410 remain inside the patient. While the illustrated process uses two anchors 302, similar processed using more anchors 302 are within the scope of the present disclosure. Also, as detailed below, in some embodiments, a single longer anchor 302 may be used.

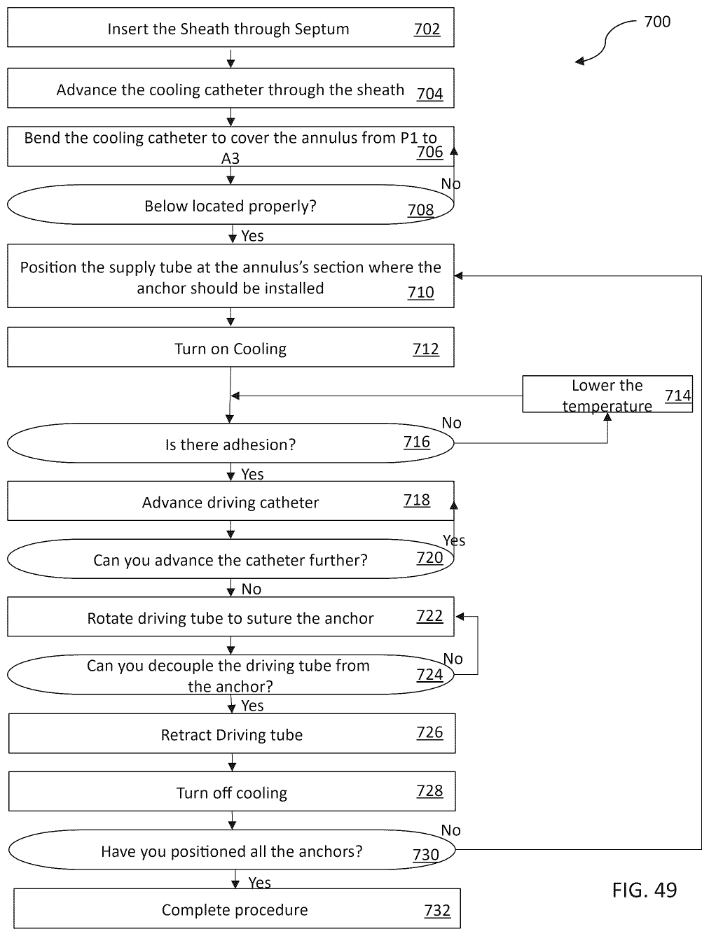

Referring to FIG. 49, the method 600 can be modified slightly to perform the alternative method 700. In the method 700, a cooling catheter 200 having a relatively long below 206 may be used. For example, the below 206 may be long enough to cover most of the targeted circumference of the annulus 611, which means that it will need to be positioned only once. This makes the procedure faster and more efficient. In this embodiment, the cooling fluid supply 214 may take the form of a tube that may be movable along the bellow 206 to selectively cool various portions thereof.

Initially, the method 700 may be like the method 702, except that the bellow 206 may be now positioned and bent to be adjacent a larger portion of the mitral valve 609, for example covering from scallop P1 to segment A3 prior to cooling (steps 702 to 708, like steps 602 to 608, but with different positioning of the bellow 206), as seen in FIG. 75.

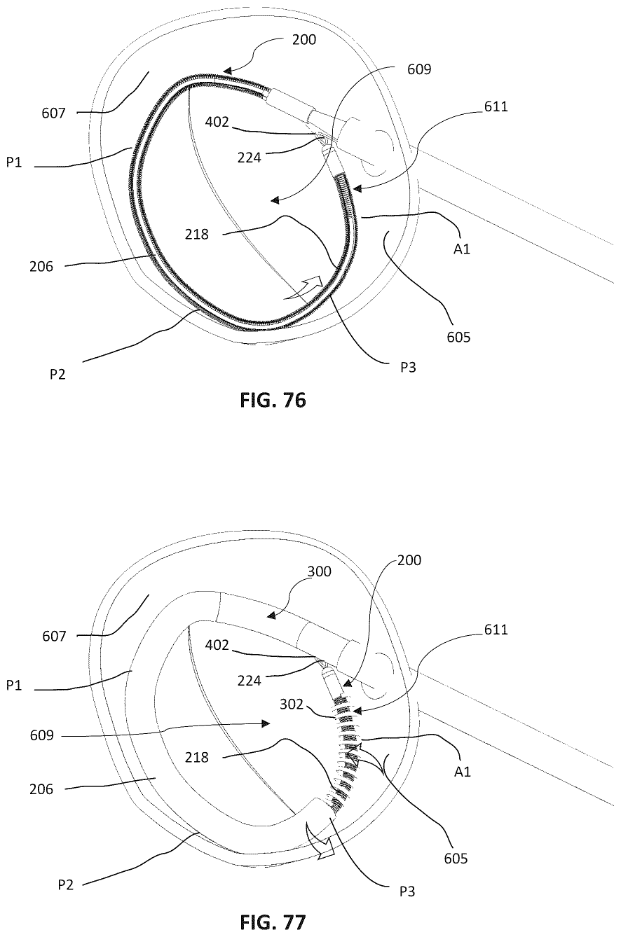

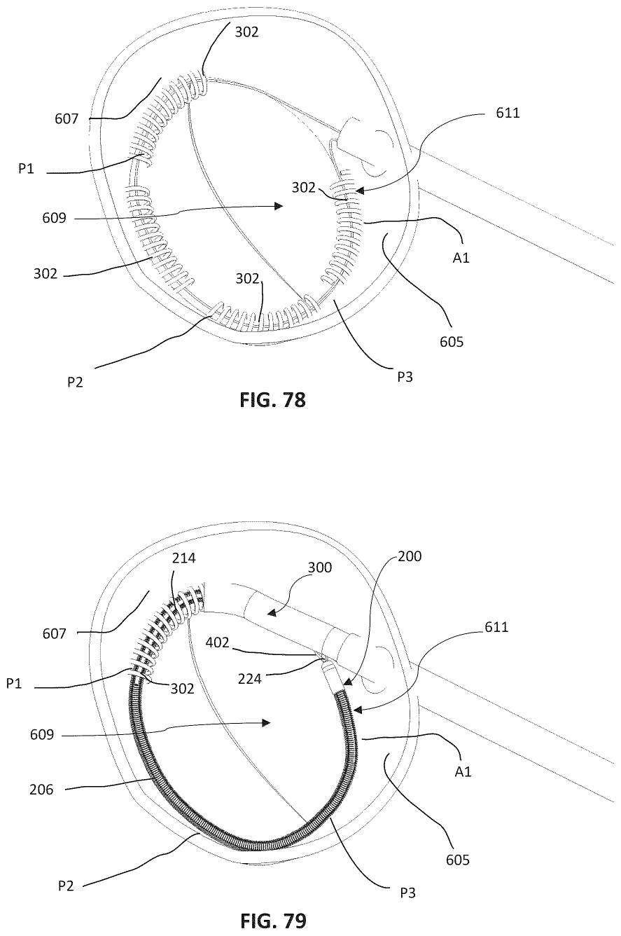

Then, the anchors 302 may be inserted one after the other, from the distalmost to the proximal most. To that effect, the cooling fluid supply 214 may be moved to a section of the bellow 206 corresponding to the location of the anchor 302 that may be driven (step 710) and cooling may be performed until there is adhesion (steps 712 to 716, like steps 610 to 614), as seen in FIG. 76. One of the anchors 302 may be now driven over the adhered portion of the bellow 206, as seen in FIG. 77 followed by termination of cooling (steps 718 to 728, like steps 616 to 626). If more anchors 302 are needed (step 730), steps 710 to 728 may be repeated at another location along the bellow 206 until all the anchors 302 have been sutured, as seen in FIG. 78. This requires only moving the cooling supply 718 along the bellow 206, instead of having to move the whole cooling catheter 200. Afterward, the procedure may be completed (step 732) similarly to steps 656 to 688.

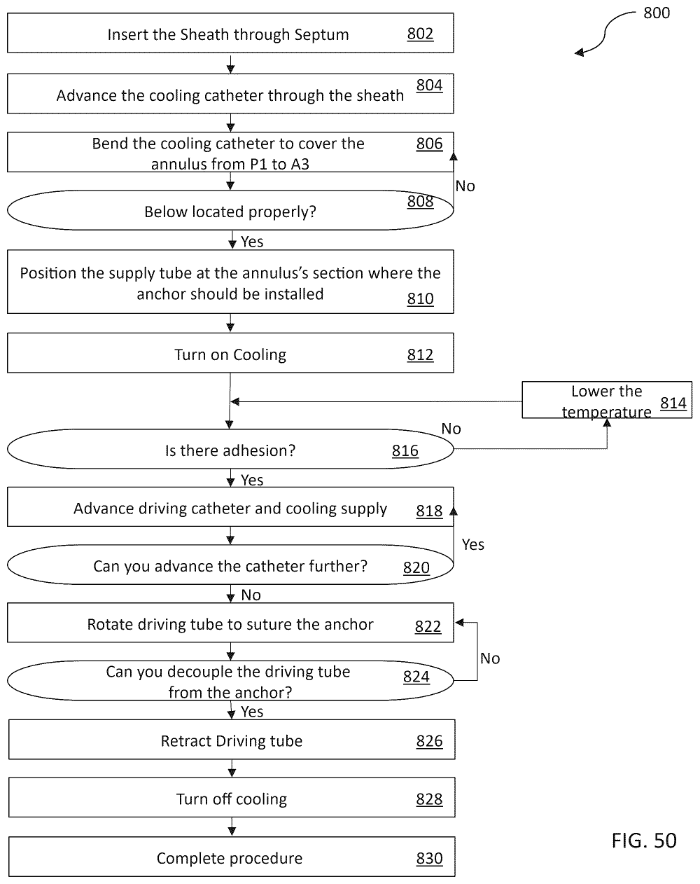

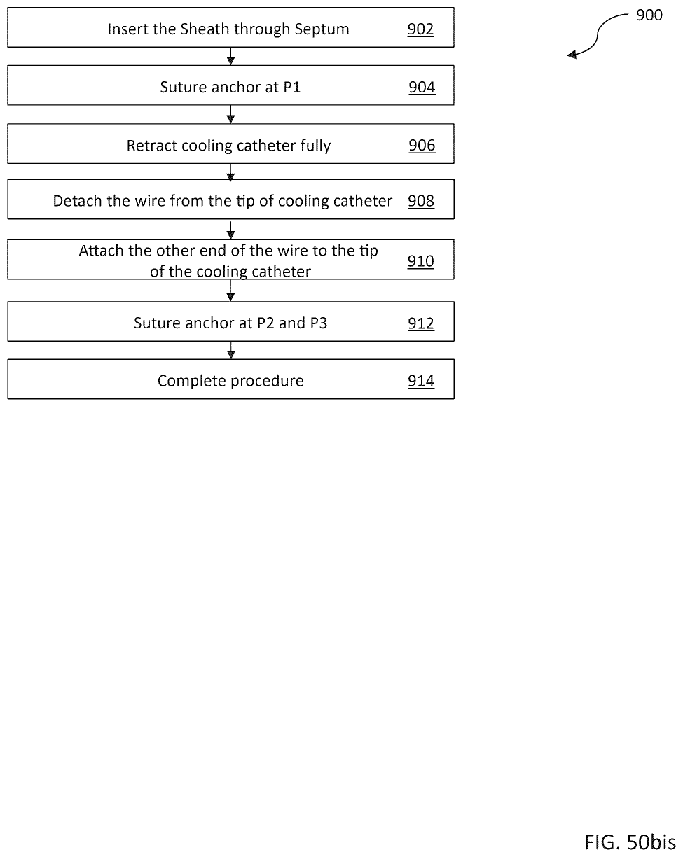

Referring to FIG. 50, in yet another method 800 using a long bellow 206, the valve repair procedure may be performed using a single long anchor 302 instead of multiple small ones. In method 800, the bellow 206 is positioned as in method 700 (steps 802 to 808, like steps 702 to 708. Once this is done, the cooling fluid supply 214 is positioned to cool the proximal portion of the bellow 206 (step 810) and cryoadhesion may be performed (steps 814 and 816), for example to adhere adjacent scallop P1.

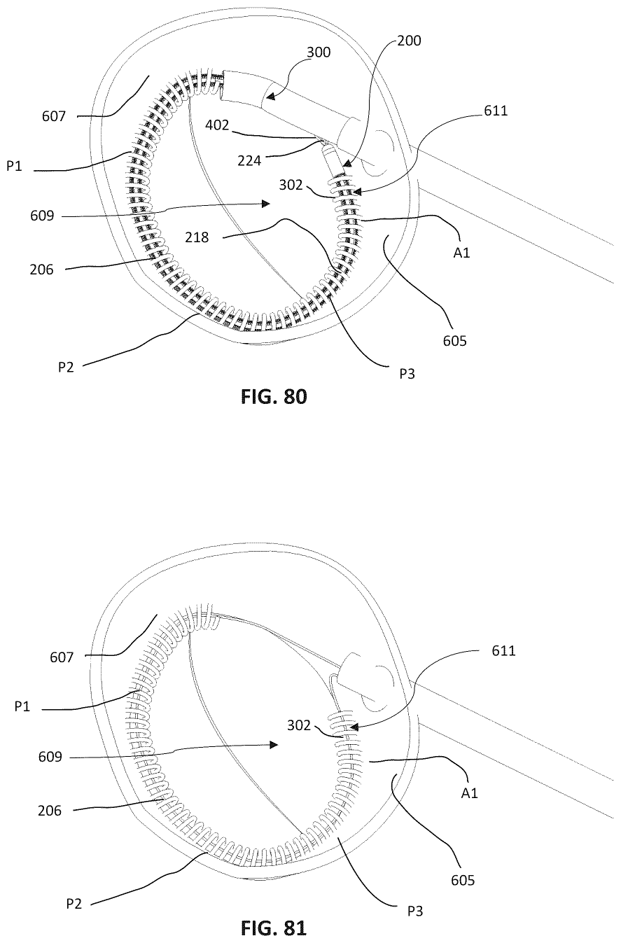

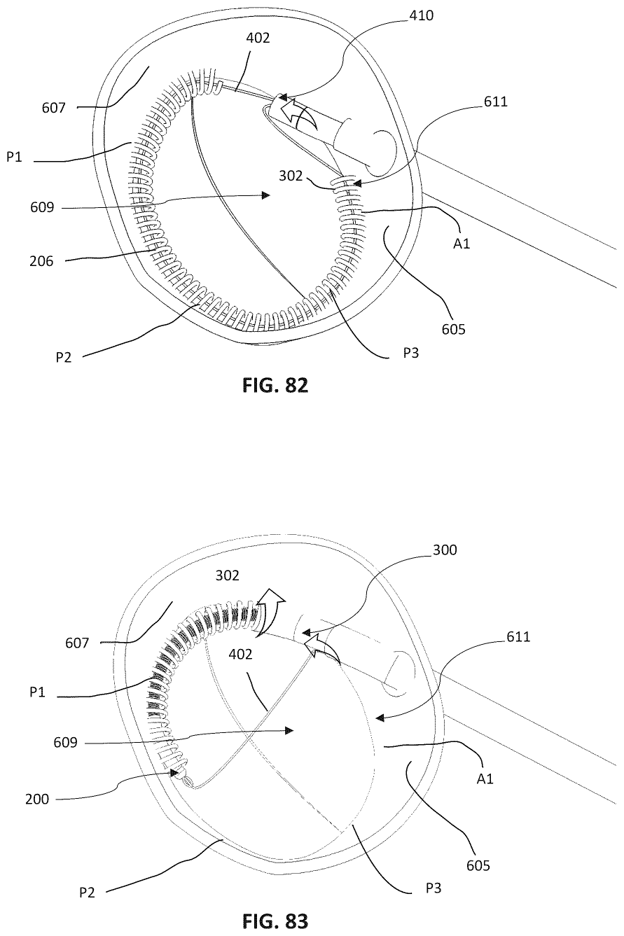

The anchor 302 can now be sutured as described above (steps 818 to 826, corresponding to steps 718 to 724), with a difference that the cooling supply 718 is also advanced as the driving catheter is advanced, to adhere the bellow 206 to tissue in which the anchor 302 is driven at its tip, as seen in the sequence of FIGS. 79 and 80. Once the anchor 302 is fully inserted, as seen in FIG. 81, the cinching procedure may be performed as a described above (steps 826 to 830, similar to steps 726 to 732), as illustrated in FIG. 82.

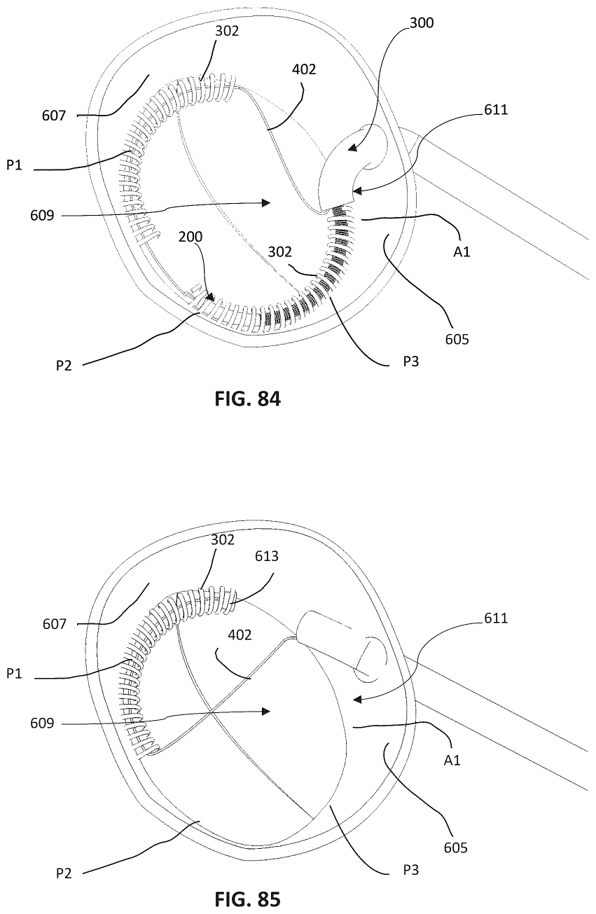

FIG. 50bis illustrates yet another method 900 of performing the annuloplasty procedure. This procedure reverses the order in which the anchors 302 may be implanted relative to the method 600. After the sheath 603 has been inserted and has traversed the septum (step 902, like step 602), one anchor 302 is anchored at scallop P1 (step 904), as illustrated in FIG. 83. The sub steps of step 904 may be like steps 604 to 626, except for the location at which the anchor 302 is inserted. Then, the cooling catheter 200 is fully retracted from the sheath (step 906) and the cinching wire 402 is detached from the wire attachment 224 (step 908) so that the opposite end of the cinching wire 402 (that was outside the patient in step 904) can be attached to the wire attachment 224 (step 910). Then, the cooling catheter 200 is inserted back in the sheath 603 so that another anchor 302 can be inserted at scallops P2 and P3 (step 912), similarly, to step 904, but at a different location. Note that in step 910, as seen in FIG. 84, the anchor 302 is driven in a direction opposite the anchors have been driven so far. That is the anchor is driven in a direction leading from scallop P3 towards scallop P1, while the opposite was true in the other anchoring steps above. Finally, the procedure is completed (step 914) by cinching using the stopper 613 and the cinching element 410, similarly to steps 656 to 688.

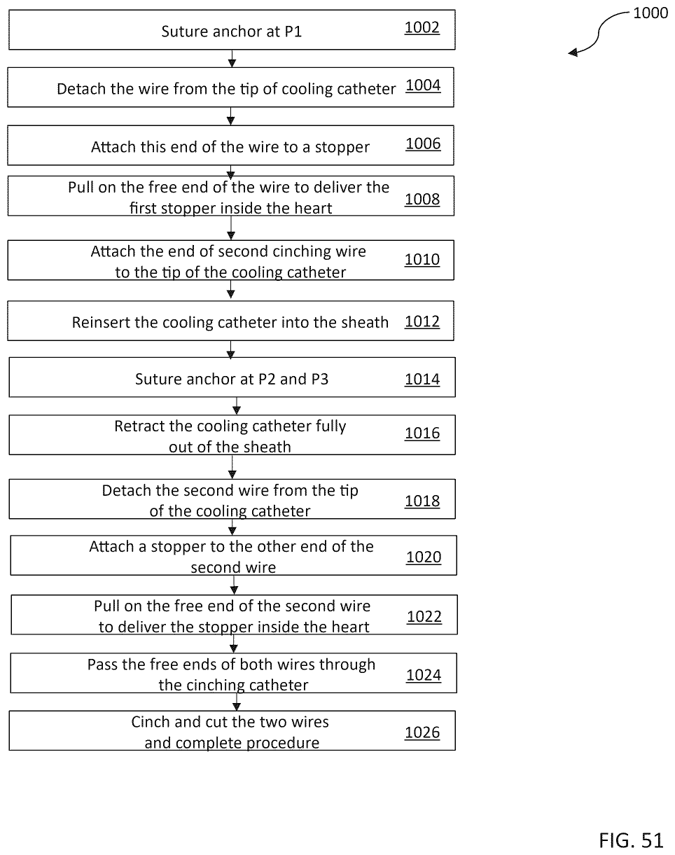

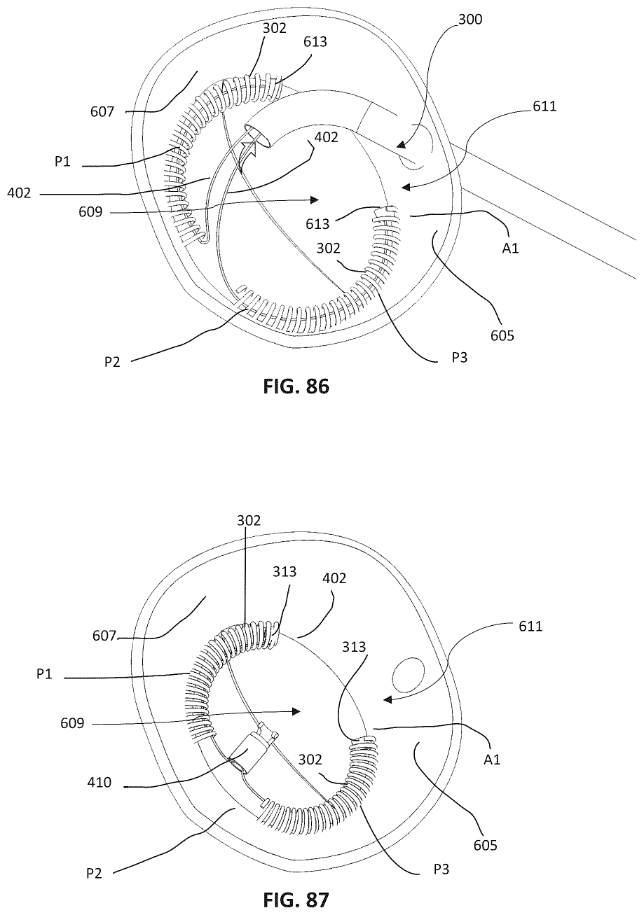

Method 1000 illustrated in FIG. 51 is an alternative to method 900. In method 1000, the anchors 302 may be sutured in the same order as in method 900, but two stoppers 613 may be used. More specifically, the anchor at scallop P1 is anchored and the cooling catheter 200 is fully retracted from the sheath 603 (step 1002) as in steps 902 and 904. Then, the cinching wire 402 is detached from the wire attachment 224 (step 1004) to be attached instead to a stopper 613 (step 1006), followed by pulling the stopper through the sheath 603 to reach the anchor 302, as seen in FIG. 85. A second cinching wire 402 is then attached to the wire attachment 224 (step 1010) and another anchor 302 is anchored as described above at scallops P2 and P3 (steps 1012 and 1014), but this time in a direction leading from scallop P2 towards scallop P3. The first cinching wire 402 remains in the sheath 603 during this process. This is followed by withdrawal of the cooling catheter 200 and insertion of a second stopper 613 (steps 1016 to 1022). After this has been performed, there are two stoppers 613 in the heart, each abutting against a respective one of the anchors 302, at opposite ends of the anchor chain, as seen in FIG. 86.

Two cinching wires 402 protrude from the sheath 603 and can be used to perform the cinching process. These two wires exit from the anchors 302 in between the anchors 302. To cinch, the two cinching wires 402 may be inserted through the cinching catheter 400 (step 1024). Then cinching and cutting of the cinching wires 402 can be performed as described above, but by working simultaneously on both cinching wires 402 (step 1026) to get finally the configuration shown in FIG. 87.

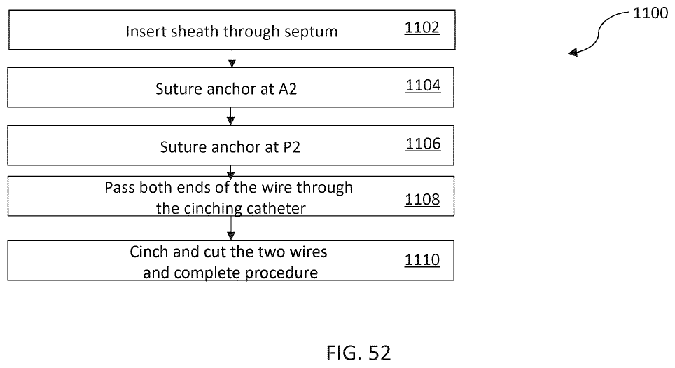

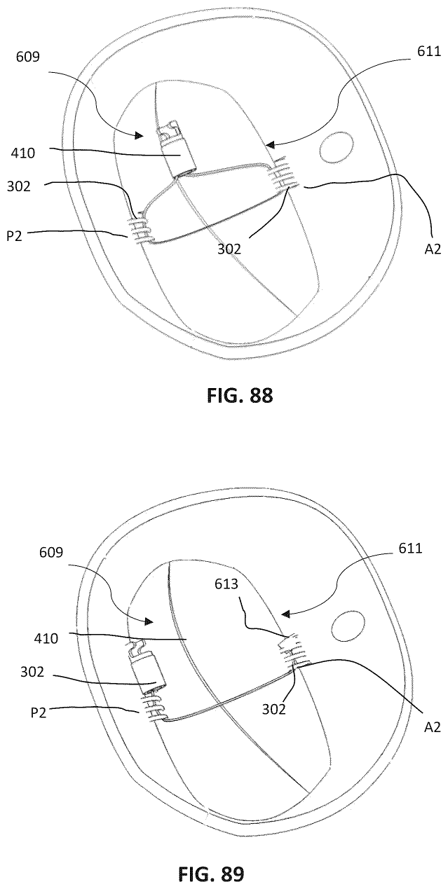

FIG. 52 illustrates another method 1100 for reducing valve regurgitation called AP reduction. Instead of using anchors anchored along scallops P1, P2 and P3, as described above, this method uses anchors 302 anchored at segment A2 and scallop P2, facing each other across the mitral valve 609. By reducing a distance between these two anchors 302, the AP distance is reduced, thereby reducing or eliminating regurgitation. In this method 1100, after insertion of the sheath through the septum (step 1102), relatively short anchors 302 are anchored successively at segment A2 (step 1104) and segment P2 (step 1106), similarly to steps 604 to 656, but at different locations. Then, both ends of the cinching wire 402 can be inserted through the cinching catheter 400 (step 1108) and cinching by pulling on both ends of the cinching wire 402 can be performed, followed by simultaneous cutting of the two ends of the cinching wire 402 (step 1110), similarly to what has been described above. By pulling enough on the two ends of the cinching wire, a length of a loop passing through both anchors 302 is reduced, which brings the valve leaflets towards each other, as seen in FIG. 88. In a variant, a stopper may be used, so that the method 1100 becomes very similar to the method 600, except for the location at which the anchors may be inserted, as illustrated in FIG. 89.

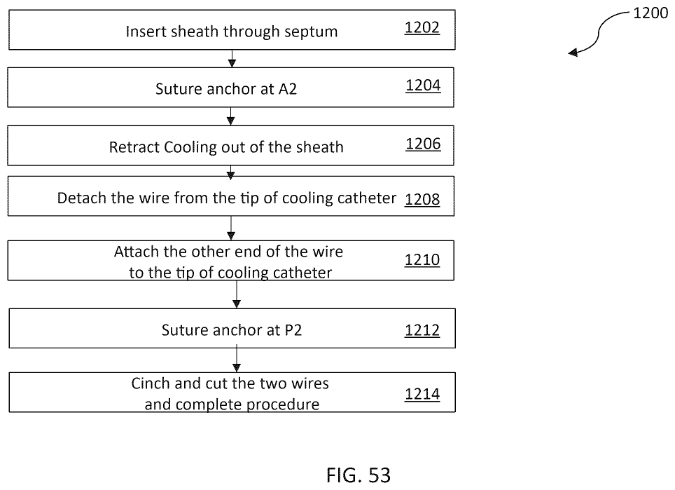

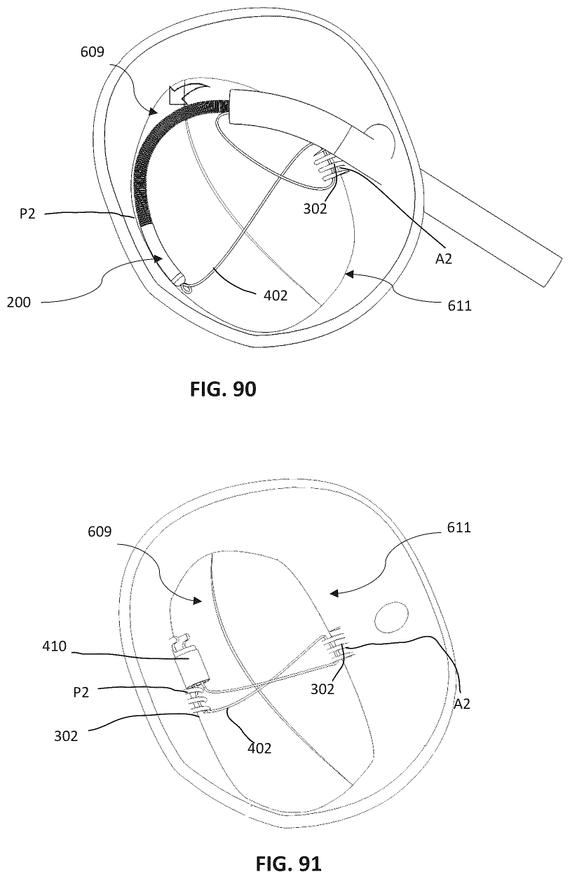

With reference to FIG. 53, in another variant, a method 1200 may be performed. In this variant, the initial steps 1202 and 1204 may be identical to steps 1102 and 1104. Then, the cooling catheter 200 is fully retracted out of the sheath 603 (step 1206) so that the cinching wire 402 can be detached from the wire attachment 224 to attach the opposite end of the cinching wire 402 to the wire attachment 224 (steps 1208 and 1210). Then, the cooling and driving catheters 200 and 300 may be used similarly to step 1106 to anchor the second anchor 302 (step 1212), as seen in FIG. 90. Afterwards, the cinching element 410 may be used as described above with both ends of the cinching wire 402 passed therethrough, and the cinching wire is cut (step 1214), similarly to step 1110. In some embodiments, the cinching element 410 is located adjacent the second anchor 302 (the one anchored adjacent scallop P2). As can be seen from FIG. 91, this creates a crossing of the cinching wire 402 across itself in the gap between the anchors 302, which may help secure the cinching wire 402.

Components of the system 100 may be used in other surgical procedures including in suturing applications, for use in endoscopic gastroscopy, and for closing internal or external wounds among other possibilities. The driving catheter 300′ is for example used to manipulate an anchor 302′ to which a suture wire 1309 may be mounted. The suture wire 1309 is typically barbed, but unbarbed suture wires 303 may be usable in some embodiments, which may require one to tie a knot to the suture wire 1309 at the end of the process to prevent the latter from unwinding. The suture wire 1309 may be terminated by a mounting tip 1313 that is removably mountable at the distal end of the anchor 302′. In such embodiments, the driving catheter 300′ may be modified with respect to the above-described driving catheter 300 in that the anchor 302′ is then permanently secured thereto.

In such embodiments, the actuator 104′, seen in FIG. 107, is like the actuator 104, except that it has a gun shape including a handle 112 with an adjacent trigger 114. The trigger 114 may be used to control supply of the cooling fluid. For example, the trigger 114 operates a valve that can selectively let pressurized or liquefied gas, such as CO2, exit from a canister housed in the handle 112. Also, the driving device 300′ like the driving catheter 300 may be used. The driving device 300′ is substantially rigid to be freely standing for external suturing, or rigid enough to be used in laparoscopy, gastroscopy, and gynecology among many other surgical procedures that require minimally invasive access. The actuator 104′ may also be provided with a slider 126 for performing functions like the slider 452 for cutting and a spool to apply tension to the suture.

In addition, it is possible to use such systems for use in a deployable fashion, for wound that are on the skin, in wound cuts for example. The cooling then advantageously provides a temporary relief of bleeding and pain, in addition to being usable for wound closure.

In another embodiment, the tip of the driving device 300′, can be attached to a hernia patch for example and used to advance the helicoid anchor 302 along tissue to attach a patch or another fabric material to tissue

It is important to note that the temperatures of cooling can be selected to sometimes kill tissue, in cases where for example bleeding can be stopped or when appropriately needed. When used externally, the cooling temperature can be lowered, for example to lower temperatures than when used internally, for example as low as −150 C.

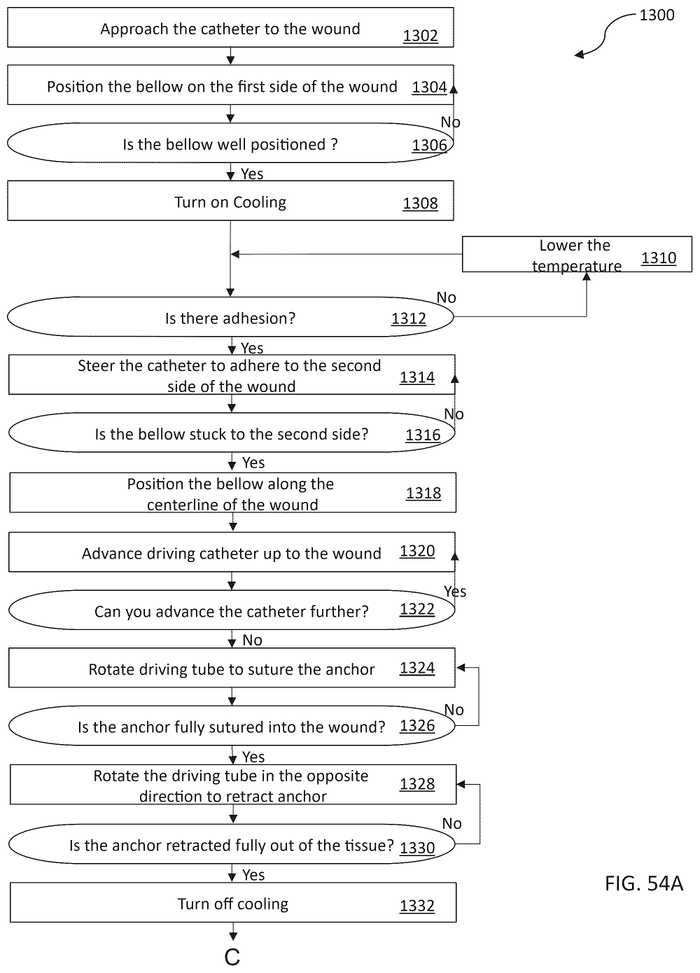

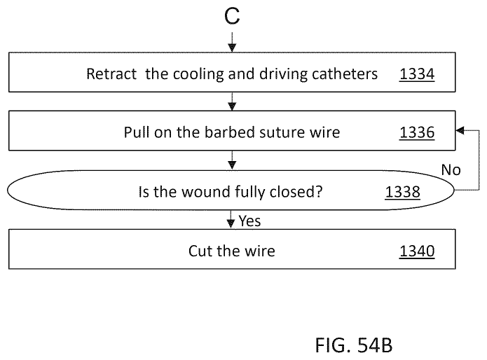

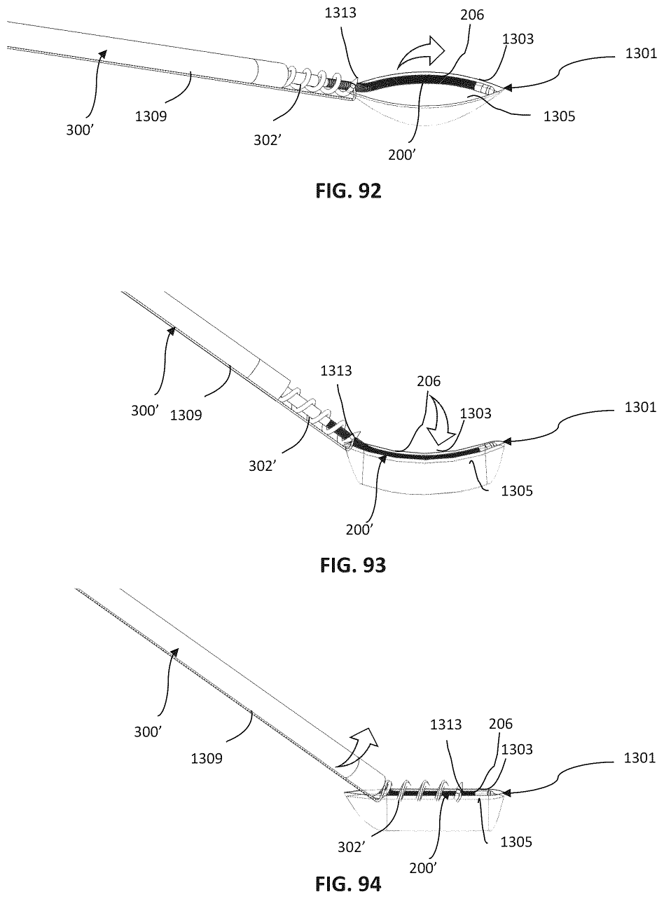

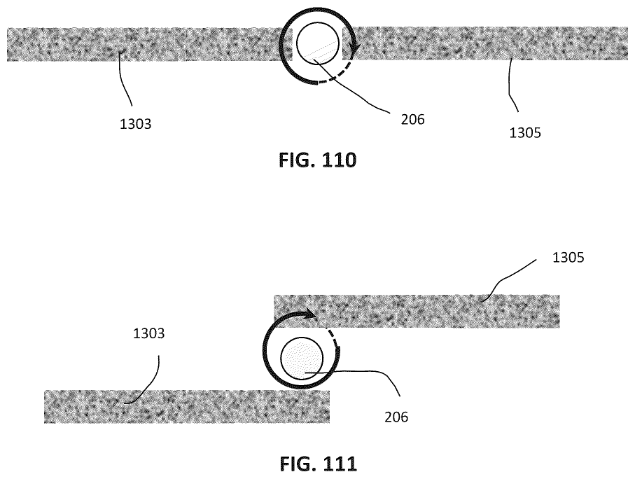

FIGS. 54A and 54B collectively illustrate an example of a method 1300 used for closing a wound 1301. First, a cooling device 200′ (like the cooling catheter 200) is approached close to the wound 1301 and positioned so that the bellow 206 abuts a first side 1303 of the wound 1301 (steps 1304 and 1306), as seen in FIG. 92. Then, the bellow 206 is cooled until the bellow 206 adheres to the tissues on the first side 1303 of the wound 1301 (steps 1310 and 1312). Subsequently, the bellow 206 is steered until it contacts the second side 1305 of the wound 1301 and adheres thereto (steps 1314 and 1316), as seen in FIG. 93. The bellow 206 is then moved to be aligned with a centerline of the wound 1301 (step 1318). It should be noted that in such applications, the bellow 206 may be replaced by any suitable similar tubular structure, such as tubing made of plastic or braided metal, among other possibilities. The first and second sides 1303 and 1305 may be positioned either side-by side, in a common plane, or to overlap at their free ends, as seen respectively in FIGS. 110 and 111.

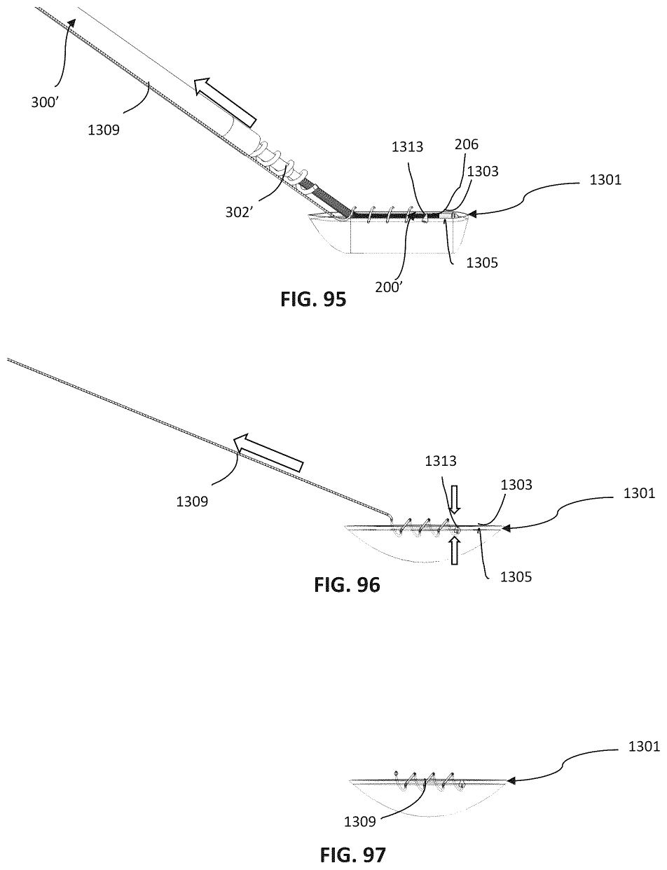

The anchor 302′ is then driven into the wound using the driving device 300′ until the anchor 302′ is fully sutured into the wound 1301 (steps 1320 to 1326), as seen in FIG. 94. To remove the anchor from the wound 1301, the driving device 300′ is rotated in the opposite direction to the direction used to drive the anchor 302′ in the tissue (steps 1328 and 1330). This is configured to result in detachment of the mounting tip 1313 from the anchor 302′ as the mounting tip 1313 is only pinned to the distal end of the anchor 302′. The suture wire 1309 is left behind by this process as the mounting tip 1313 is shaped to be blunt opposed to its driving tip, as seen in FIG. 95. Cooling can then stop (step 1332) so that the cooling and driving devices 200 and 300′ can be removed (step 1334). Pulling on the barbed suture wire 1309, as seen in FIG. 96, with then close the wound 1301 completely (step 1336 and 1338). The wound 1301 will remain closed even after releasing the tension in the wire 1301 due to the barbs digging into the tissue and not allowing the wire lose tension. Finally, the suture wire 1309 can be cut flush with the wound 1301 to get a fully sutures wound 1301, as seen in FIG. 97. If an unbarbed suture wire 1309 is used, one may have to tie a knot at the end of the suture wire 1309 before cutting to prevent the suture wire 1303 from unwinding.

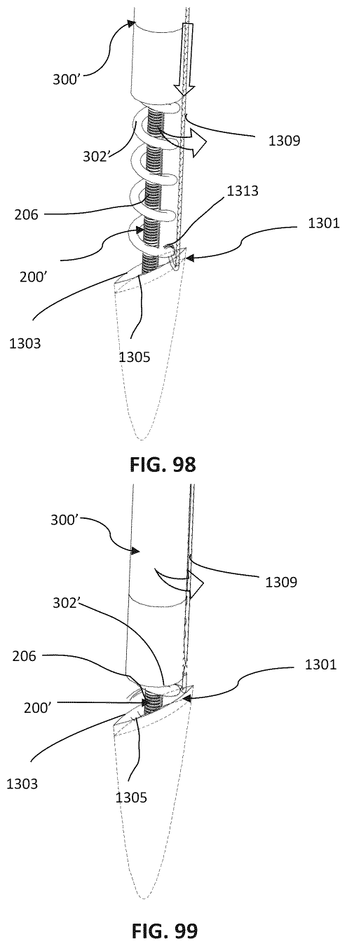

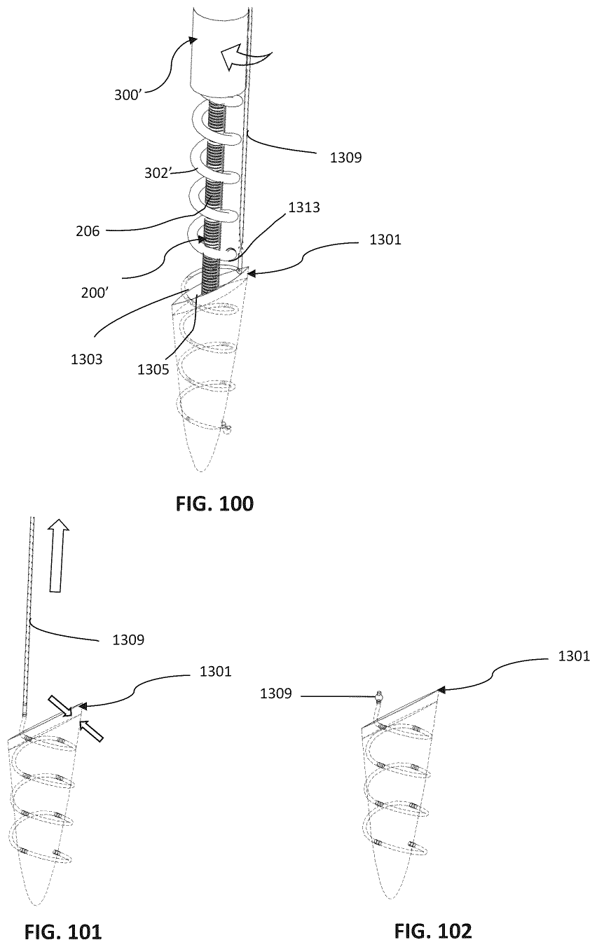

If the wound 1301 is deep and narrow, instead of being shallower and longer, one can insert the bellow 206 along the depth of the wound instead and perform the same steps. This process is shown in FIGS. 98 to 102, which correspond respectively to FIGS. 93 to 97 described hereinabove, with the difference therebetween being due to the different orientation of the anchor 302.

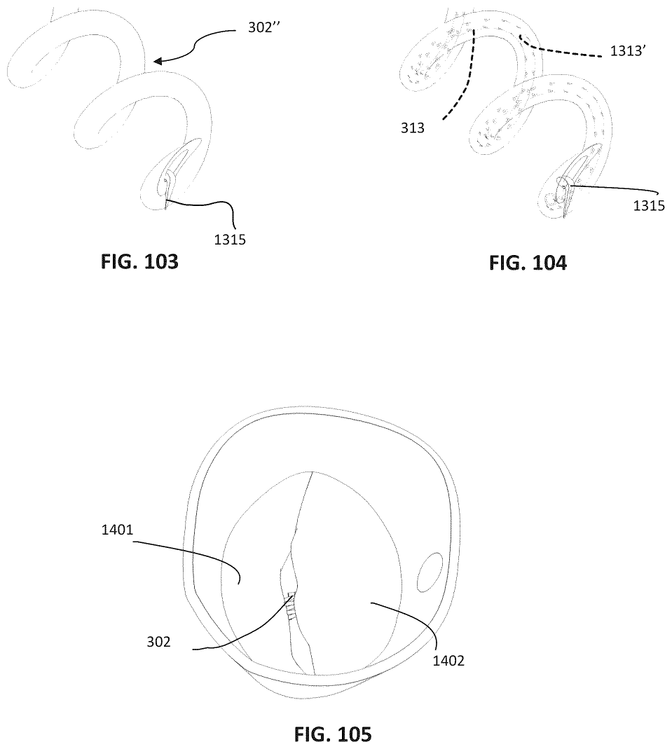

In some embodiments, as seen in FIGS. 103 and 104 a hollow anchor 302″ defining an internal passageway 313 may be used. The internal passageway is formed inside the helical structure of the hollow anchor 302″ and is therefore also helical in shape. The suture wire 1313′ extends through the internal passageway 313 and may be terminated by a hook 1315 that is configured to engage the tissues when the anchor 302″ is withdrawn. The hook 1315 may be larger than the diameter of the internal passageway 313, so that it remains at the tip of the anchor 302″ when the latter is driven. The anchor 302″ may also be only partially hollow.

As seen in FIG. 1, in some embodiments a tensioning device 116 may be used to adjust tension of the cinching wire 400 when the cooling and driving catheters 200 and 300 may be used. The tensioning device may include a spool and works similarly to the spool 454 of the cinching catheter 400.

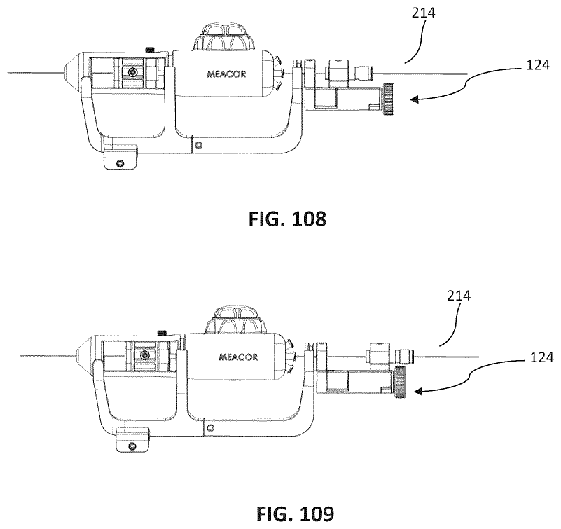

As seen in FIGS. 108 and 109, in some embodiments the cooling fluid supply 214 may be mounted to a fluid supply actuator allowing precise positioning of the cooling fluid supply 214. For example, the cooling fluid supply 214 may be clamped to a clamp that is movable along a guide fixed relative to the actuator 104. Movements of the clamp along the guide can be controlled for example by a worm drive operated by a knob, among other possibilities.

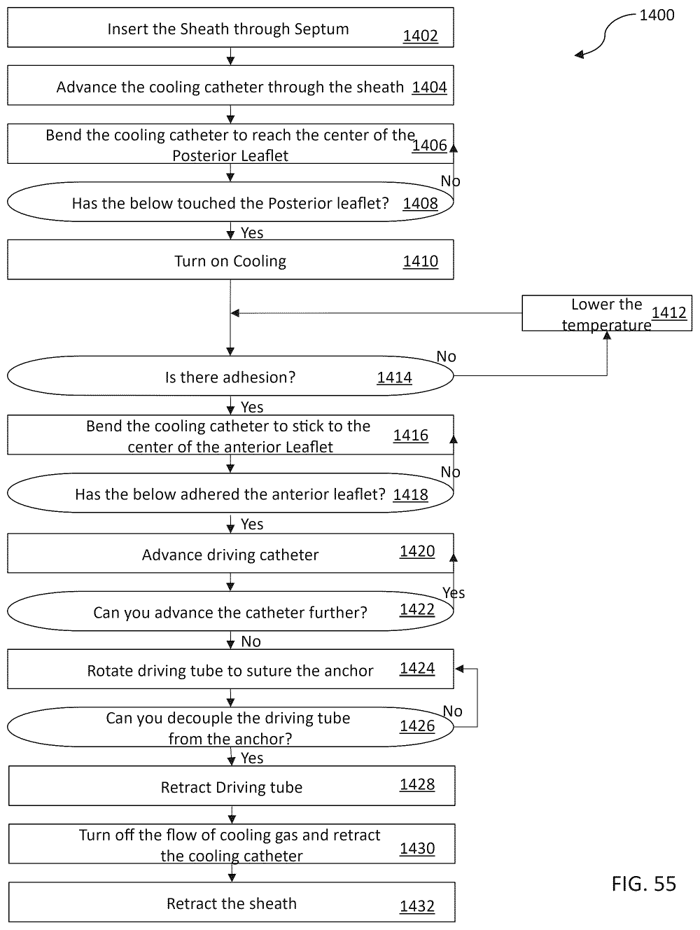

Another manner of mitigating mitral valve 609 regurgitation is to perform an edge-to-edge valve repair. With reference to FIG. 55, a method 1400 to perform this procedure is shown. The system 100 can be used to that effect similarly to the suture process described above, except that no suture wire may be used. Instead, an anchor 302 may be used to bind the central portion of both leaflets of the mitral valve 609.

More specifically, the sheath 603 may be inserted through the septum (step 1402). Then, the cooling catheter 200 may be slid inside the sheath 603 and the bellow 206 may be bent and positioned at the center of the posterior leaflet 1401 (steps 1404, 1406 and 1408). When a sufficient position is achieved, the flow of cooling gas may be turned on to decrease the temperature of the bellow to allow it to stick to the leaflet through cryoadhesion (steps 1410, 1412 and 1414). The flow of cooling gas may continue until the anchor 302 is fully positioned in place.

The cooling catheter 200 and sheath 603 may be then steered so that the bellow 206 touches the center of the anterior leaflet 1403 (while still stuck to the posterior leaflet 1401) (steps 1416 and 1418). This will enable the bellow 206 to stick to the anterior leaflet 1413 as well. Then, the driving catheter 300 may be advanced and the anchor 302 may be driven through both the anterior and posterior leaflets 1403 and 1401, similarly to the above-described anchoring procedures, therefore suturing both leaflets to each other (steps 1420, 1422, 1424 and 1426). Once the anchor 302 is fully deployed, the driving catheter 300 can be removed (step 1428) and cooling can stop (step 1430) to allow retraction of the cooling catheter 200 and then the sheath 603 (steps 1432) to obtain the result shown in FIG. 105.

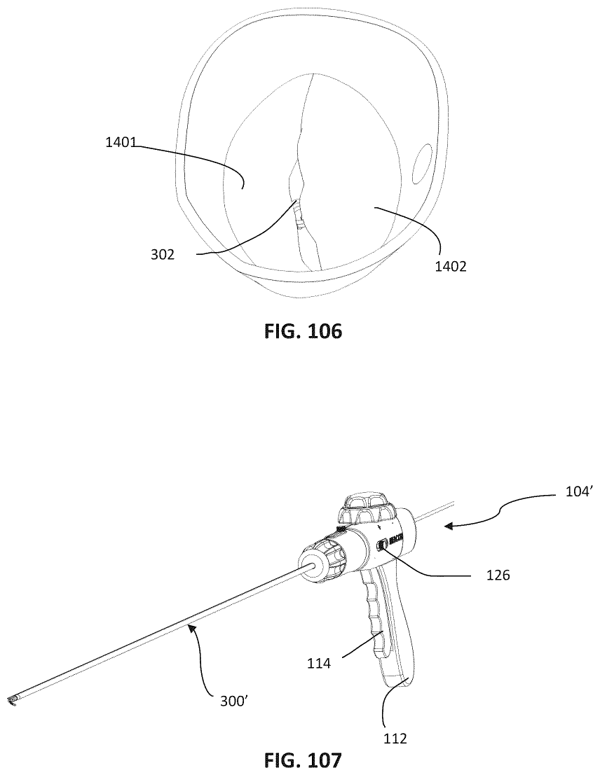

A similar procedure can be followed to insert the anchor perpendicularly to the plane of the mitral valve 609. In this variant, the bellow 206 is inserted perpendicular to the mitral valve 609 and adhered to the anterior and posterior leaflets 1403 and 1401 to allow driving of the anchor 302 therethrough to get the result shown in FIG. 106. In another variant, the anchor may be inserted perpendicularly to the mitral valve 609, as described above, but the anchor may include a suture wire. Retracting the anchor, similarly to method 1200, sutures the anterior and posterior leaflets 1403 and 1401 to each other, which can be followed by cutting of the wire. This procedure may be therefore like the wound closing method but performed inside the heart to attach the anterior and posterior leaflets to each other.

While manually operated actuator have been illustrated, powered actuators including electric motors or other powered actuating mechanisms may be also used.

The foregoing discussion discloses and describes merely exemplary embodiments of the disclosure. One skilled in the art will readily recognize from such discussion and from the accompanying drawings and claims, that various changes, modifications and variations can be made therein without departing from the spirit and scope of the disclosure as defined in the following embodiments and claims.

Embodiment 1. A cutting catheter for cutting a wire, the cutting catheter comprising: an elongated wire receiving member provided with a wire receiver at a wire receiving member distal end thereof for receiving the wire; an elongated cutting member provided with an annular blade at a cutting member distal end thereof, the annular blade being positionable adjacent the wire receiver; the cutting member being longitudinally movable relative to the wire receiving member and axially rotatable relative thereto so that the blade can both translate and rotate relative to a wire received in the wire receiver.

Embodiment 2. The cutting catheter as defined in embodiment 1, wherein the cutting member may be biased in a distally oriented direction relative to the wire receiver.

Embodiment 3. The cutting catheter as defined in embodiment 1, wherein longitudinal movements and axial rotations of the cutting member relative to the wire receiving member may be independent from each other.

Embodiment 4. The cutting catheter as defined in embodiment 1 or 2, wherein the wire receiving and cutting members are flexible.

Embodiment 5. The cutting catheter as defined in embodiment 1, 2 or 3, wherein the wire receiving and cutting members are bendable to be able follow a patient's vasculature.

Embodiment 6. The cutting catheter as defined in embodiment 1 to 5, wherein the wire receiver may include a wire aperture extending transversally therethrough for receiving the wire therethrough.

Embodiment 7. The cutting catheter as defined in embodiment 6, wherein the wire receiving member defines a wire receiving member passageway extending longitudinally therethrough and opening in the wire aperture; and the wire receiving member and cutting member are concentric.

Embodiment 8. The cutting catheter as defined in embodiment 6 or 7, further comprising a tensioner for applying a tension to the wire.

Embodiment 9. The cutting catheter as defined in embodiment 1 to 8, wherein the cutting member may include a tubular member surrounding at least part of the wire receiving member.

Embodiment 10. The cutting catheter as defined in embodiment 1 to 9, further comprising an actuating assembly, the actuating assembly including a body, the wire receiving member being mounted to the body; a cutting member actuator operatively coupled to the cutting member for selectively and independently rotating and longitudinally moving the cutting member relative to the body.

Embodiment 11. The cutting catheter as defined in embodiment 10, wherein the cutting member may be movable between proximal and distal positions relative to the body, the cutting member being lockable in translation relative to the body in the proximal position.

Embodiment 12. The cutting catheter as defined in embodiment 11, further comprising a biasing element provided between the cutting member and the body for biasing the cutting member towards the distal position.

Embodiment 13. The cutting catheter as defined in embodiment 10, 11 or 12, wherein the cutting member actuator may include a cutting member mount, the cutting member extending through the cutting member mount and being jointly axially rotatable therewith; and a knob mounted to the body so as to be axially rotatable relative thereto, the knob defining a knob aperture extending axially therethrough receiving the cutting member mount so that the cutting member mount may be axially movable along the knob and jointly rotatable with the cutting member mount.

Embodiment 14. A method of cutting a wire using a tubular cutting member provided with a distal annular blade, the method comprising: positioning the annular blade to abut against the wire at a contact location with the wire entering the cutting member; and axially rotating the annular blade to cut through the wire.

Embodiment 15. The method as defined in embodiment 14, further comprising pushing the annular blade towards the wire.

Embodiment 16. The method as defined in embodiment 15, wherein pushing the annular blade towards the wire may include biasing the annular blade with a biasing element.

Embodiment 17. The method as defined in embodiment 14, 15 or 16, wherein the annular blade may be rotated over at least one full turn before the wire is cut.

Embodiment 18. The method as defined in embodiment 14 to 17, wherein the wire may be pinched between the cutting blade and a member fixed relative to the annular blade.

Embodiment 19. The method as defined in embodiment 14 to 18, further comprising applying a tension on the wire.

Embodiment 20. The method as defined in embodiment 14 to 19, wherein the method may be performed inside a body of a patient.

Embodiment 21. A cinching system usable with a beaded wire defining beads, comprising a cinching element including an attachment; and a stopper configured for allowing movements of the beaded wire therethrough in a distal to proximal direction and prevent movements of the beaded wire therethrough in a proximal to distal direction; and a cinching catheter including a distally provided cinching element holder for receiving the attachment; and a cinching element lock for reversibly locking the attachment and the cinching element holder to each other.

Embodiment 22. The cinching system as defined in embodiment 21, wherein the stopper may include a tubular body and leaves provided inside the tubular body and converging towards each other inside the stopper in a direction leading proximally, the leaves being movable between narrow and wide configurations, a central gap between the leaves being smaller in the narrow configuration than in the wide configuration so that the beads are movable through the gap in the wide configuration but prevented from moving through the gap in the narrow configuration.

Embodiment 23. The cinching system as defined in embodiment 22, wherein the leaves extend integrally as a single piece of material from the tubular body.

Embodiment 24. The cinching system as defined in embodiment 22 or 23, wherein the leaves are made of a Ni—Ti shape memory alloy.

Embodiment 25. The cinching system as defined in embodiment 23 or 24, wherein, in the wide configuration, the leaves are biased towards the narrow configuration.

Embodiment 26. The cinching system as defined in embodiment 23 to 25, wherein the cinching catheter may be adapted to selectively open the gap to allow both distally and proximally oriented movements of the beaded wire therethrough.

Embodiment 27. The cinching system as defined in embodiment 26, wherein the tubular body may include circumferentially spaced apart sectors, the sectors being deformable between undeformed and an open configuration wherein, in the open configuration the gap larger than in the undeformed configuration; the cinching catheter may include a sector actuator for selectively moving the sectors to the open configuration.

Embodiment 28. The cinching system as defined in embodiment 21 to 27, wherein the cinching element holder and the attachment are complementarily shaped so that when the cinching element holder and the attachment are mounted to each other: relative longitudinal and circumferential movements between the cinching element holder and the attachment are prevented; and unless the attachment is locked to the cinching element holder, the attachment may be free to move laterally in at least one direction relative to the cinching element holder.

Embodiment 29. The cinching system as defined in embodiment 28, wherein the attachment and cinching element holder are both hollow; and the cinching element lock may include an elongated member selectively movable between extended and retracted positions, wherein in the extended position, the cinching element lock extends through both the cinching element holder and the attachment; and in the retracted position, the cinching element lock is retracted proximally relative to the attachment so that the latter is free to move laterally relative to the cinching element holder.

Embodiment 30. The cinching system as defined in embodiment 29, wherein the attachment and cinching element holder are both internally threaded and the cinching element lock may be externally threaded, so that in the extended position, the cinching element lock threadedly engages the attachment and cinching element holder.

Embodiment 31. The cinching system as defined in embodiment 30, wherein the stopper may be hollow and may include deformable leaves extending internally and configured to allow passage of the beads through the stopper in a proximally leading direction while preventing passage of the beads through the stopper in a distally leading direction.

Embodiment 32. The cinching system as defined in embodiment 31, wherein the stopper may include a tubular stopper body defining circumferential sectors separated from each other by slits, each leaf extending from a single sector; the cinching element lock may be hollow and movable distally to engage the sectors to flare the sectors outwardly and separate the leaves from each other to create a gap allowing the beads to move distally therethrough.

Embodiment 33. The cinching system as defined in embodiment 28, wherein the cinching element lock may be tubular and selectively movable between extended and retracted positions, wherein in the extended position, the cinching element lock surrounds at least part of both the cinching element holder and the attachment to prevent lateral movements therebetween; and in the retracted position, the cinching element lock may be retracted proximally relative to the attachment so that the latter may be free to move laterally relative to the cinching element holder.

Embodiment 34. The cinching system as defined in embodiment 28 to 33, wherein the leaves together form a conical shape tapering proximally.

Embodiment 35. A cinching element defining proximal and distal ends, the cinching element being usable with a cinching catheter and a beaded wire, the cinching element comprising: a proximally located attachment selectively attachable to the cinching catheter; and a distally located stopper configured for allowing movements of the beaded wire therethrough in a distal to proximal direction and prevent movements of the beaded wire therethrough in a proximal to distal direction.

Embodiment 36. The cinching element as defined in embodiment 35, wherein the stopper may include a tubular body and leaves provided inside the tubular body and converging towards each other inside the stopper in a direction leading proximally, the leaves being movable between narrow and wide configurations, a central gap between the leaves being smaller in the narrow configuration than in the wide configuration so that the beads are movable through the gap in the wide configuration but prevented from moving through the gap in the narrow configuration.

Embodiment 37. The cinching element as defined in embodiment 36, wherein the leaves extend integrally as a single piece of material from the tubular body.

Embodiment 38. The cinching element as defined in embodiment 37, wherein the leaves are made of a Ni—Ti shape memory alloy.

Embodiment 39. The cinching element as defined in embodiment 36 to 38 wherein, in the wide configuration, the leaves are biased towards the narrow configuration.

Embodiment 40. The cinching element as defined in embodiment 36 defining circumferentially spaced apart sectors, the leaves being each supported by one of the sectors, the sectors being deformable to an open configuration wherein the gap may be enlarged to allow movements of the beads in the proximal to distal direction.

Embodiment 41. A cryoadhesion procedure catheter assembly, comprising: a tubular body supporting a hollow thermally transmissive element distally relative thereto, the thermally transmissive element including a flexible portion bendable to conform to a predetermined shape; a pulling assembly secured distally to the thermally transmissive element and including a pull wire extending along the body, the pulling assembly being configured for bending the flexible portion; and a cooling fluid supply for supplying a cooling fluid to the thermally transmissive element.

Embodiment 42. The cryoadhesion procedure catheter assembly as defined in embodiment 41, further comprising a guiding member extending inside the flexible portion, the guiding member being stiffer in bending in a first plane than in a second plane orthogonal to the first plane, both the first and second planes extending along the bellow, wherein the guiding member limits bending in the first plane when the pull wire may be pulled while allowing bending in the second plane.

Embodiment 43. The cryoadhesion procedure catheter assembly as defined in embodiment 42, wherein the guiding member may include a plate extending laterally across the flexible member and longitudinally along at least part of the flexible member.

Embodiment 44. The cryoadhesion procedure catheter assembly as defined in embodiment 43, wherein the plate may be perforated.

Embodiment 45. The cryoadhesion procedure catheter assembly as defined in embodiment 41 to 44, wherein the cooling fluid supply defines a fluid outlet, the cooling fluid supply being movable relative to the thermally transmissive portion so that the cooling fluid outlet may be positionable at different longitudinal positions there along.

Embodiment 46. The cryoadhesion procedure catheter assembly as defined in embodiment 41 to 45, further comprising an anchor driving catheter for driving a helical anchor over the thermally transmissive portion.

Embodiment 47. The cryoadhesion procedure catheter assembly as defined in embodiment 46, wherein the tubular body defines an anchor engagement proximally to the thermally transmissive portion, the anchor engagement being configured for engaging the anchor and constraining movements of the anchor there along to a helical movement.

Embodiment 48. The cryoadhesion procedure catheter assembly as defined in embodiment 47, wherein the anchor engagement prevents the anchor driving catheter from advancing distally further than the anchor engagement.

Embodiment 49. The cryoadhesion procedure catheter assembly as defined in embodiment 48, wherein the anchor engagement may include at least two protrusions extending radially outwardly from the tubular body circumferentially and longitudinally offset relative to each other.

Embodiment 50. The cryoadhesion procedure catheter assembly as defined in embodiment 48, wherein the anchor engagement may include a helical flange protruding radially outwardly from the tubular body.

Embodiment 51. The cryoadhesion procedure catheter assembly as defined in embodiment 46 to 50, further comprising an anchor catheter actuator for selectively advancing and rotating the anchor driving catheter relative to the tubular body.

Embodiment 52. The cryoadhesion procedure catheter assembly as defined in embodiment 51, wherein advancement and rotation of the anchor driving catheter are independent from each other.

Embodiment 53. The cryoadhesion procedure catheter assembly as defined in embodiment 52, wherein advancement and rotation of the anchor driving catheter are lockable independently from each other.

Embodiment 54. The cryoadhesion procedure catheter assembly as defined in embodiment 53, wherein the anchor catheter actuator may include a casing and a knob mounted to the casing, the knob defining an axial knob passageway receiving the driving catheter therethrough, the driving catheter being secured to the knob to be jointly axially movable and rotatable relative thereto.

Embodiment 55. The cryoadhesion procedure catheter assembly as defined in embodiment 54, wherein the knob may be mounted to a knob mount to be jointly longitudinally movable therewith and axially rotatable relative thereto, the knob mount being mounted to the casing to be longitudinally movable there along and fixed in axial rotation relative thereto.

Embodiment 56. The cryoadhesion procedure catheter assembly as defined in embodiment 55, further comprising a rotation lock for selectively locking a relative rotation between the knob mount and the knob and a translation lock for selectively locking a relative translation between the casing and the knob mount.

Embodiment 57. The cryoadhesion procedure catheter assembly as defined in embodiment 56, wherein the casing defines a longitudinally elongated mount cavity receiving the knob mount thereinto and a pair of slits extending longitudinally there along between outside of the casing and the mount cavity, the translation and rotation locks including respectively a translation lock threaded fastener and a rotation lock threaded fastener each extending radially through a respective one of the slits so as to be longitudinally movable there along and engaging a respective threaded aperture formed in the knob mount, wherein when the translation and rotation lock threaded fasteners are fully threaded in their respective threaded aperture, translation and rotation of the knob relative to the casing are respectively locked.

Embodiment 58. A transcatheter system, comprising: a driving catheter configured to drive one or more anchors into tissue; a cinching catheter configured to cinch a wire extending through the one or more anchors; and a cutting catheter configured to cut a portion of the wire.

Embodiment 59. The transcatheter system as defined in embodiment 58, further comprising a cooling catheter and a cooling system for providing a cooling fluid to the cooling catheter, wherein the cooling catheter may be configured to cryoadhere to a portion of the tissue.

Embodiment 60. The transcatheter system as defined in embodiment 59, wherein the cooling catheter may include a thermally transmissive portion having a flexible portion, the flexible portion being bendable to conform to a shape of a valve annulus.

Embodiment 61. The transcatheter system as defined in embodiment 59, wherein, when bent, the flexible portion extends over between about 135 and about 225 degrees.

Embodiment 62. The transcatheter system as defined in embodiment 58 to 61, further comprising a tensioning device for applying tension to the wire.

Embodiment 63. The transcatheter system as defined in embodiment 58 to 62, wherein the wire defines longitudinally spaced apart beads.

Embodiment 64. The transcatheter system as defined in embodiment 58 to 63, further comprising at least one helical anchor mountable to the driving catheter for being driven in the tissue.

Embodiment 65. The transcatheter system as defined in embodiment 64, further comprising a stopper and a cinching element, the stopper and cinching element being both larger than a central passageway of the helical anchor, the wire being securable to the stopper and the cinching element being configured to allow passage of the wire therethrough only in one direction.

Embodiment 66. The transcatheter system as defined in embodiment 64, wherein the at least one anchor may be configured to be implanted at a mitral valve annulus along a portion of the mitral valve selected from the group consisting of: from the P1 area to the A1 area, from the P2 area to the A3 area, from the P1 area to the P3 area, at the P1 area, at the P2 area, at the P3 area, from the P3 to the A3 area.

Embodiment 67. The transcatheter system as defined in embodiment 64, wherein the at least one anchor may include from 2 to 30 coils.

Embodiment 68. The transcatheter system as defined in embodiment 64, wherein the at least one anchor is configured to span between about 45 degrees and about 225 degrees along the mitral valve annulus.

Embodiment 69. The transcatheter system as defined in embodiment 64, wherein the at least one anchor is configured to span between about 225 degrees and about 315 degrees along the mitral valve annulus.

Embodiment 70. The transcatheter system as defined in embodiment 58 to 69, wherein the cinching catheter may include a double shell construction including two layers axially rotatable relative to each other and configured for receiving the wire therebetween.

Embodiment 71. The transcatheter system as defined in embodiment 59, wherein the cooling and driving catheter are the cryoadhesion procedure catheter assembly as defined in any one of embodiments 41 to 57, the cutting catheter may be an in any one of embodiments 1 to 20 and the cinching catheter may be as defined in any one of embodiments 21 to 40.

Embodiment 72. An implant kit, comprising: a helical anchor defining an anchor passageway extending therethrough; a beaded wire defining longitudinally spaced apart beads; a stopper, the stopper being larger than the central passageway to be prevented from passing therethrough; and a cinching element larger than the central passageway to be prevented from passing therethrough, the cinching element defining a one-way gap allowing passage of the wire therethrough in only one direction.

Embodiment 73. The implant kit as defined in embodiment 72, wherein the stopper may be securable to the beaded wire.

Embodiment 74. The implant kit as defined in embodiment 72, wherein the kit may be assembled to form an implant wherein the stopper and cinching element abut against the helical anchor at opposed longitudinal ends thereof with the wire extending therebetween under tension.

Embodiment 75. The implant kit as defined in embodiment 72, further comprising another helical anchor, wherein the kit may be assembled to form an implant wherein the helical anchors are in prolongation of each other with a space therebetween and with the stopper and cinching element abutting against a respective one of the helical anchors opposed to the space with the wire extending therebetween under tension.

Embodiment 76. The implant kit as defined in embodiment 72, wherein the anchor may be rectilinear when undeformed and curved when the implant is assembled with the wire under tension.

Embodiment 77. The implant kit as defined in embodiment 72, wherein the stopper may be securable to the wire, the implant kit further comprising another stopper securable to the wire, another helical anchor, and another beaded wire.

Embodiment 78. The implant kit as defined in embodiment 77, wherein the kit may be assembled to form an implant wherein the helical anchors are in prolongation of each other with a space therebetween with the stoppers abutting against a respective one of the helical anchors opposed to the space, each wire extending from a respective one of the stoppers through a respective helical anchor and reaching the cinching element provided adjacent the space, the wires both extending under tension through the gap.

Embodiment 79. An implant kit, comprising at least two helical anchors defining each an anchor passageway extending therethrough; a beaded wire defining longitudinally spaced apart beads; a cinching element defining a one-way gap allowing passage of the wire therethrough in only one direction.

Embodiment 80. The implant kit as defined in embodiment 79, wherein the kit may be assembled to form an implant wherein the wire forms a loop extending through both helical anchors and closed by the cinching element.

Embodiment 81. The implant kit as defined in embodiment 80, wherein the loop forms an 8 figure.

Embodiment 82. The implant kit as defined in embodiment 81, wherein in the implant, the two helical anchors are laterally spaced apart from each other with substantially parallel anchor passageways.

Embodiment 83. An implant assembled from the implant kit according to any one of embodiments 72 to 83.

Embodiment 84. A wound closing device, comprising: a cryoadhesion device including a hollow thermally conductive element and a cooling fluid supply for supplying a cooling fluid thereto; and an anchor driver for driving a helical anchor over the thermally conductive element.

Embodiment 85. The wound closing device as defined in embodiment 84, wherein the anchor driver supports the anchor at a distal end of a rigid member.

Although the present disclosure has been described hereinabove by way of exemplary embodiments thereof, it will be readily appreciated that many modifications are possible in the exemplary embodiments without materially departing from the novel teachings and advantages of this present disclosure. Accordingly, the scope of the claims should not be limited by the exemplary embodiments but should be given the broadest interpretation consistent with the description as a whole. The present disclosure can thus be modified without departing from the spirit and nature of the subject invention as defined in the appended claims.

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-40. (canceled)

41. A cryoadhesion procedure catheter assembly, comprising:
a tubular body supporting a hollow thermally transmissive element distally relative thereto, the thermally transmissive element including a flexible portion bendable to conform to a predetermined shape;
a pulling assembly secured distally to the thermally transmissive element and including a pull wire extending along the body, the pulling assembly being configured for bending the flexible portion; and
a cooling fluid supply for supplying a cooling fluid to the thermally transmissive element.

42. The cryoadhesion procedure catheter assembly as defined in claim 41, further comprising a guiding member extending inside the flexible portion, the guiding member being stiffer in bending in a first plane than in a second plane orthogonal to the first plane, both the first and second planes extending along the bellow, wherein the guiding member limits bending in the first plane when the pull wire is pulled while allowing bending in the second plane.

43-44. (canceled)

45. The cryoadhesion procedure catheter assembly as defined in claim 41, wherein the cooling fluid supply defines a fluid outlet, the cooling fluid supply being movable relative to the thermally transmissive portion so that the cooling fluid outlet is positionable at different longitudinal positions there along.

46. The cryoadhesion procedure catheter assembly as defined in claim 41, further comprising an anchor driving catheter for driving a helical anchor over the thermally transmissive portion.

47. The cryoadhesion procedure catheter assembly as defined in claim 46, wherein the tubular body defines an anchor engagement proximally to the thermally transmissive portion, the anchor engagement being configured for engaging the anchor and constraining movements of the anchor there along to a helical movement.

48. The cryoadhesion procedure catheter assembly as defined in claim 47, wherein the anchor engagement prevents the anchor driving catheter from advancing distally further than the anchor engagement.

49. The cryoadhesion procedure catheter assembly as defined in claim 48, wherein the anchor engagement includes at least two protrusions extending radially outwardly from the tubular body circumferentially and longitudinally offset relative to each other.

50. The cryoadhesion procedure catheter assembly as defined in claim 48, wherein the anchor engagement includes a helical flange protruding radially outwardly from the tubular body.

51. The cryoadhesion procedure catheter assembly as defined in claim 46, further comprising an anchor catheter actuator for selectively advancing and rotating the anchor driving catheter relative to the tubular body.

52. The cryoadhesion procedure catheter assembly as defined in claim 51, wherein advancement and rotation of the anchor driving catheter are independent from each other.

53. The cryoadhesion procedure catheter assembly as defined in claim 52, wherein advancement and rotation of the anchor driving catheter are lockable independently from each other.

54-57. (canceled)

58. A transcatheter system, comprising:
a driving catheter configured to drive one or more anchors into tissue;
a cinching catheter configured to cinch a wire extending through the one or more anchors; and
a cutting catheter configured to cut a portion of the wire.

59. The transcatheter system as defined in claim 58, further comprising a cooling catheter and a cooling system for providing a cooling fluid to the cooling catheter, wherein the cooling catheter is configured to cryoadhere to a portion of the tissue.

60. The transcatheter system as defined in claim 59, wherein the cooling catheter includes a thermally transmissive portion having a flexible portion, the flexible portion being bendable to conform to a shape of a valve annulus.

61. The transcatheter system as defined in claim 59, wherein, when bent, the flexible portion extends over between about 135 and about 225 degrees.

62. The transcatheter system as defined in claim 58, further comprising a tensioning device for applying tension to the wire.

63. The transcatheter system as defined in claim 58, wherein the wire defines longitudinally spaced apart beads.

64. The transcatheter system as defined in claim 58, further comprising at least one helical anchor mountable to the driving catheter for being driven in the tissue.

65. The transcatheter system as defined in claim 64, further comprising a stopper and a cinching element, the stopper and cinching element being both larger than a central passageway of the helical anchor, the wire being securable to the stopper and the cinching element being configured to allow passage of the wire therethrough only in one direction.

66. The transcatheter system as defined in claim 64, wherein the at least one anchor is configured to be implanted at a mitral valve annulus along a portion of the mitral valve selected from the group consisting of: from the P1 area to the A1 area, from the P2 area to the A3 area, from the P1 area to the P3 area, at the P1 area, at the P2 area, at the P3 area, from the P3 to the A3 area.

67-89. (canceled)
